(19) United States
(12) Patent Application Publication
(10) Pub. No.: US 2024/0010978 A1
(43) Pub. Date: Jan. 11, 2024

(54) METHOD FOR PREPARING DENDRITIC CELL USING PLATELET LYSATE

(71) Applicants: ALP REGENERATIVE MEDICINE INSTITUTE, INC., Kanazawa-shi, Ishikawa (JP); KANAZAWA MEDICAL UNIVERSITY, Kahoku-gun, Ishikawa (JP)

(72) Inventors: Shigetaka SHIMODAIRA, Kahoku-gun (JP); Terutsugu KOYA, Kahoku-gun (JP); Takuya SAKAMOTO, Kahoku-gun (JP); Misa TOGI, Kahoku-gun (JP)

(73) Assignees: ALP REGENERATIVE MEDICINE INSTITUTE, INC., Kanazawa-shi, Ishikawa (JP); KANAZAWA MEDICAL UNIVERSITY, Kahoku-gun, Ishikawa (JP)

(21) Appl. No.: 18/035,123

(22) PCT Filed: Nov. 4, 2021
(86) PCT No.: PCT/JP2021/040505

§ 371 Date: May 3, 2023

(30) Foreign Application Priority Data
Nov. 4, 2020 (JP) 2020-184317

(51) Int. Cl. C12N 5/0784; A61K 35/15

(52) U.S. Cl. CPC C12N 5/0639; A61K 35/15; C12N 2506/115; C12N 2500/84

(57) ABSTRACT

This invention provides a method for preparing dendritic cells from monocytes using a platelet lysate. This invention also provides a method for preparing cytotoxic dendritic cells from monocytes comprising performing non-adhesion culture of monocytes separated from the peripheral blood using a serum-free medium containing a human platelet lysate (HPL), GM-CSF, and PEGylated interferon α and performing further non-adhesion culture with the addition of prostaglandin E2 and OK432.

### TECHNICAL FIELD

The present invention relates to a method for preparing dendritic cells from monocytes.

### BACKGROUND ART

Dendritic cells (DCs) are strong antigen presenting cells in vivo and DCs are known to present antigens to T cells to induce immune responses. DCs are also known to react directly, not only with T cells, but also with B cells, NK cells. NK T cells, and other cells, and play a key role in immune responses. When immature DCs receive antigenic stimulation, DCs acquire the high capacity for T cell stimulation while elevating expression levels of CD40, CD80, CD83, and the like, migrate to the peripheral lymphoid tissue, and activate T cells specific to the antigens incorporated therein to induce immune responses.

In general, several types of cytokines are recognized as substances capable of inducing differentiation of dendritic cells from blood progenitor cells. For example, there are many reports concerning induction of DC differentiation with the use of GM-CSF in combination with IL-4 (Non-Patent Document 1). There is another report concerning substances capable of induction of DC differentiation by itself or in combination with other cytokines (Non-Patent Document 2). Examples of reported substances include TNF-α, IL-2, IL-3, IL-6, IL-7, IL-12, IL-13, IL-15, HGF (hepatocyte growth factor), CD40 ligand, M-CSF, Flt3 ligand, c-kit ligand, and TGF-β.

A method for inducing DC differentiation with the use of GM-CSF in combination with IL-4 comprises adhesion culture in which mononuclear cells (monocytes and lymphocytes) are seeded in a culture dish, lymphocytes are washed, and adhered monocytes are used for culture. Culture is performed in the presence of GM-CSF/IL-4 for 5 to 7 days, cells are collected by washing in a medium and scraping (physical stripping), the medium is exchanged with a fresh medium containing an adjuvant (immunostimulator) OK432, and mature DCs are thus prepared.

In addition, a method for preparing dendritic cells using G-CSF (Patent Document 1) and a method for preparing dendritic cells via non-adhesion culture using IFN (Patent Document 2) had been reported.

### PRIOR ART DOCUMENTS

### Patent Documents

Patent Document 1: WO 2014/126250
Patent Document 2: WO 2016/148179

### Non-Patent Documents

Non-Patent Document 1: Akagawa K. S. et al., Blood, Vol. 88, No. 10 (November 15), 1996: pp. 4029-4039
Non-Patent Document 2: O'Neill D. W., et a., Blood, Vol. 104, No. 8 (October 15), 2004: pp. 2235-2246

### SUMMARY OF THE INVENTION

### Objects to Be Attained by the Invention

The present invention provides a method for preparing dendritic cells from monocytes using a platelet lysate.

### Means for Attaining the Objects

Methods for preparing dendritic cells (DCs) from monocytes in the peripheral blood had heretofore been reported, although conventional methods were not sufficient in respect of DC yield. In addition, DCs having cytotoxic activity or other activity in addition to a strong antigen presenting ability or phagocytic ability had been desired for the purpose of use of DCs for cancer treatment.

The present inventors have conducted concentrated studies in order to enhance DC yield and prepare DCs with high functionality. As a result, they discovered that the optimized DCs could be prepared within a short period of time, a yield of DC preparation could be enhanced, and DCs with strong cytotoxicity could be obtained with the use of a platelet lysate (HPL), GM-CSF, and PEGylated interferon α by separating monocytes from the peripheral blood and subjecting the monocytes to non-adhesion culture; i.e., suspension culture, to prepare DCs. This has led to the completion of the present invention.

Specifically, the present invention is as described below.

[1] A method for preparing cytotoxic dendritic cells from monocytes comprising performing non-adhesion culture of monocytes separated from the peripheral blood with the use of a serum-free medium containing a human platelet lysate (HPL), GM-CSF, and PEGylated interferon α and performing further non-adhesion culture with the addition of prostaglandin E2 and OK432.
[2] The method for preparing dendritic cells from monocytes according to [1] comprising performing non-adhesion culture with the use of a serum-free medium containing a human platelet lysate (HPL), GM-CSF, and PEGylated interferon α for 2 to 5 days and performing further culture with the addition of prostaglandin E2 and OK432 for 1 to 2 days.
[3] The method for preparing dendritic cells from monocytes according to [1] or [2] comprising culturing monocytes with the use of a serum-free medium containing a 1 to 10 (v/v) % human platelet lysate (HPL), 100 U/ml to 10,000 U/ml GM-CSF, 500 ng/ml to 5 μg/ml PEGylated interferon α, 5 ng/ml to 50 ng/ml prostaglandin E2, and 5 μg/ml to 50 μg/ml OK432.
[4] The method for preparing dendritic cells from monocytes according to any of [1] to [3], wherein the serum-free medium is DCO-K.
[5] The method for preparing dendritic cells from monocytes according to any of [1] to [4], wherein the viability of the dendritic cells is 90% or higher and the yield, which is the proportion of the number of the dendritic cells relative to the number of monocytes during culture, is 15% or higher.
[6] The method for preparing dendritic cells from monocytes according to any of [1] to [5], wherein the dendritic cells are positive for CD14, CD16, CD56, CD83, CD86, CCR7 (CD197), HLA-ABC, and HLA-DR.
[7] Dendritic cells obtained by the method for preparing dendritic cells from monocytes according to any of [1] to [6].
[8] A pharmaceutical composition comprising the dendritic cells according to [7].
[9] The pharmaceutical composition according to [8], which has antitumor immune activity and can be used for cancer treatment.
[10] A method for separating monocytes comprising culturing peripheral blood mononuclear cells in an adhesion culture vessel with the use of a serum-free medium containing a human platelet lysate (HPL) for 15 minutes to 3 hours, removing non-adherent cells, and collecting adherent cells.
[11] The method for separating monocytes according to [10], which involves the use of a serum-free medium containing a 1 to 10 (v/v) % human platelet lysate (HPL).
[12] The method for separating monocytes according to [10] or [11], wherein the serum-free medium is DCO-K.
[13] An agent for inducing differentiation of cytotoxic dendritic cells from monocytes, which contains a human platelet lysate (HPL), GM-CSF, PEGylated interferon α, prostaglandin E2, and OK432.
[14] The agent for inducing differentiation of cytotoxic dendritic cells from monocytes according to [13], which contains an agent for inducing differentiation of immature dendritic cells containing a human platelet lysate (HPL), GM-CSF, and PEGylated interferon α and an agent for dendritic cell maturation containing prostaglandin E2 and OK432.
[15] The method according to any of [1] to [6] comprising adding a cancer-specific antigen to prepare dendritic cells having cancer-antigen-specific cytotoxic activity on dendritic cells.
[16] Dendritic cells obtained by the method according to [15] having cancer-antigen-specific cytotoxic activity on dendritic cells.
[17] A pharmaceutical composition containing the dendritic cells according to [16], which has antitumor immune activity and can be used for cancer treatment.

This description includes part or all of the content as disclosed in the description and/or drawings of Japanese Patent Application No. 2020-184317, which is a priority document of the present application.

### Effects of the Invention

According to the method for preparing dendritic cells (DCs) of the present invention comprising performing non-adhesion culture of the separated monocytes in the presence of HPL, GM-CSF. PEGylated interferon (IFN)-α (PEG-IFN-α), prostaglandin E2 (PGE2), and OK432, DCs having strong cytotoxic activity can be obtained at high yield within a short period of time. The DCs obtained can be preferably used for cancer immunotherapy.

### BRIEF DESCRIPTION OF THE DRAWINGS

FIG. 1 shows a protocol of Preliminary Test 1.

FIG. 2-1 shows images of cell morphology observation on Day 1 of Preliminary Test 1.

FIG. 2-2 shows images of cell morphology observation on Day 2 of Preliminary Test 1.

FIG. 3 shows the results of detection of cell surface antigens of IFN-DCs prepared with the use of a DCO-K medium only by flow cytometry using label antibodies in Preliminary Test 1.

FIG. 4 shows the results of detection of cell surface antigens of IFN-DCs prepared with the use of a DCO-K+ABS medium by flow cytometry using label antibodies in Preliminary Test 1.

FIG. 5 shows the results of detection of cell surface antigens of IFN-DCs prepared with the use of a DCO-K+HPL medium by flow cytometry using label antibodies in Preliminary Test 1.

FIG. 6 shows the results of detection of cell surface antigens of IFN-DCs prepared with the use of an AIM-V medium by flow cytometry using label antibodies in Preliminary Test 1.

FIG. 7 shows the results of evaluation of the purity and the contamination rate of lymphocyte at the time of IFN-DC collection by flow cytometry in Preliminary Test 1.

FIG. 8 shows a summary of the cell viability and the yield in Preliminary Test 1.

FIG. 9 shows a protocol of Preliminary Test 2.

FIG. 10 shows images of cell morphology observation of Preliminary Test 2.

FIG. 11 shows the results of detection of cell surface antigens of IFN-DCs prepared with the use of a DCO-K medium only by flow cytometry using label antibodies in Preliminary Test 2.

FIG. 12 shows the results of detection of cell surface antigens of IFN-DCs prepared with the use of a DCO-K+ABS medium by flow cytometry using label antibodies in Preliminary Test 2.

FIG. 13 shows the results of detection of cell surface antigens of IFN-DCs prepared with the use of a DCO-K+HPL medium by flow cytometry using label antibodies in Preliminary Test 2.

FIG. 14 shows the results of evaluation of the purity and the contamination rate of lymphocyte at the time of IFN-DC collection by flow cytometry using label antibodies in Preliminary Test 2.

FIG. 15 shows a summary of the cell viability and the yield in Preliminary Test 2.

FIG. 16 shows a protocol of Preliminary Test 3.

FIG. 17 shows images of cell morphology observation of Preliminary Test 3.

FIG. 18 shows the results of detection of cell surface antigens of IFN-DCs when cultured in HPL 5 (v/v) % by flow cytometry using label antibodies in Preliminary Test 3.

FIG. 19 shows the results of detection of cell surface antigens of IFN-DCs when cultured in HPL 2.5 (v/v) % by flow cytometry using label antibodies in Preliminary Test 3.

FIG. 20 shows the results of evaluation of the purity and the contamination rate of lymphocyte at the time of IFN-DC collection by flow cytometry in Preliminary Test 3.

FIG. 21 shows a summary of the cell viability and the yield in Preliminary Test 3.

FIG. 22 shows a protocol of Preliminary Test 4.

FIG. 23 shows the cell viability, the yield, and the contamination rate of lymphocyte when IFN-DCs are prepared using DCO-K media each supplemented with HPL at various concentrations (0 (v/v) %, 1 (v/v) %, 5 (v/v) %, and 10 (v/v) %) in Preliminary Test 4.

FIG. 24 shows the results of phenotype evaluation of IFN-DCs prepared with the use of HPL at various concentrations (0 (v/v) %, 1 (v/v) %, 5 (v/v) %, and 10 (v/v) %) in Preliminary Test 4.

FIG. 25 shows the results of detection of cell surface antigens of IFN-DCs when cultured in HPL at 10 (v/v) % by flow cytometry using label antibodies in Preliminary Test 4.

FIG. 26 shows a protocol of Preliminary Test 5.

FIG. 27-1 shows images of cell morphology observation and compositions of maturation cocktails of Preliminary Test 5.

FIG. 27-2 shows the results of evaluation of the contamination rate of lymphocyte at the time of IFN-DC collection by flow cytometry in Preliminary Test 5.

FIG. 28 shows the cell viability, the yield, and the contamination rate of lymphocyte when IFN-DCs are prepared using maturation cocktails in Preliminary Test 5.

FIG. 29 shows the results of phenotype analysis of IFN-DCs prepared using maturation cocktails in Preliminary Test 5.

FIG. 30 shows a protocol of Preliminary Test 6.

FIG. 31 shows the results of cytotoxic activity assays of HPL-IFN-DCs prepared using fresh or cryopreserved PBMCs in Preliminary Test 6 (Case 1).

FIG. 32 shows the results of cytotoxic activity assays of HPL-IFN-DCs prepared using fresh or cryopreserved PBMCs in Preliminary Test 6 (Case 2).

FIG. 33 shows a protocol of Preliminary Test 7.

FIG. 34 shows the results of flow cytometry analysis of the cytotoxic T cell-inducing ability of HPL-IFN-DCs prepared in serum-free media (AIM-V) in Preliminary Test 7.

FIG. 35 shows a protocol of Main Test 1.

FIG. 36-1 shows images of cell morphology observation of Main Test 1.

FIG. 36-2 shows the cell viability, the yield, and the purity of IFN-DCs and HPL-IFN-DCs collected after maturation in Main Test 1.

FIG. 37 shows the results of flow cytometry analysis of the influence of HPL on the IFN-DC phenotype in Main Test 2.

FIG. 38 shows a protocol of Main Test 3.

FIG. 39 shows the antigen phagocytic ability and the antigen degradation ability of IFN-DCs and HPL-IFN-DCs examined in Main Test 3.

FIG. 40 shows a protocol of Main Test 4.

FIG. 41 shows the results of assays of cytokines (IL-10, TGF-β, IFN-γ, TNF-α, IL-12 (p70), and IL-6) involved in induction of cytotoxic T cells secreted from HPL-IFN-DCs in Main Test 4.

FIG. 42 shows a protocol of Main Test 5.

FIG. 43-1 shows the results of flow cytometric detection of MART-1-specific cytotoxic T cells on Day 14 and Day 21 of coculture of CD8⁺ T cells with IFN-DCs and HPL-IFN-DCs subjected to pre-pulsing with MART-1 peptides in Main Test 5.

FIG. 43-2 shows the number of MART-1-specific CD8⁺ T cells when CD8⁺ T cells were cocultured with IFN-DCs and HPL-IFN-DCs subjected to pre-pulsing with MART-1 peptides in Main Test 5.

FIG. 43-3 shows the proportion of MART-1-specific CD8⁺ T cells when CD8⁺ T cells were cocultured with IFN-DCs and HPL-IFN-DCs subjected to pre-pulsing with MART-1 peptides in Main Test 5.

FIG. 44 shows the results of comparison of the cytotoxic T cell-inducing ability between IFN-DCs and HPL-IFN-DCs in Main Test 5 (Part 1).

FIG. 45 shows the results of comparison of the cytotoxic T cell-inducing ability between IFN-DCs and HPL-IFN-DCs in Main Test 5 (Part 2).

FIG. 46 shows the results of comparison of the cytotoxic T cell-inducing ability between IFN-DCs and HPL-IFN-DCs in Main Test 5 (Part 3).

FIG. 47 shows a protocol of Main Test 6.

FIG. 48-1 shows spot images demonstrating the antigen-specific IFN-γ-producing ability of cytotoxic T cells induced by IFN-DCs and HPL-IFN-DCs.

FIG. 48-2 shows the antigen-specific IFN-γ-producing ability of cytotoxic T cells induced by IFN-DCs and HPL-IFN-DCs in terms of the amount of IFN-γ production.

FIG. 49 shows a summary demonstrating that HPL-IFN-DCs are excellent in terms of the cell viability, the rate of collection, and the purity.

FIG. 50 shows a summary of HPL-IFN-DC traits.

FIG. 51 shows the results of evaluation of HPL-IFN-DC functions.

FIG. 52 shows a method for preparing IFN-DCs using HPL.

FIG. 53 shows conditions of monocytes subjected to selective adhesion culture when preparing IFN dendritic cells using HPL.

FIG. 54 shows the results of flow cytometry of monocytes subjected to selective adhesion culture when preparing IFN dendritic cells using HPL.

FIG. 55 shows the results of analysis of the HPL-IFN-DC phenotype.

FIG. 56 shows induction of MART-1 antigen-specific cytotoxic T cells by IFN-DCs or HPL-IFN-DCs.

FIG. 57 shows a protocol of the WT1-CTL induction test.

FIG. 58 shows a method for preparing IL-4-DCs (FIG. 58A) and HPL-IFN-DCs (FIG. 58B) used for the WT1-CTL induction test.

FIG. 59 shows the results of comparison of the WT1-CTL inducing ability by IL-4-DCs or HPL-IFN-DCs supplemented with WT1.

FIG. 60 shows the total number of WT1-CTL cells induced by IL-4-DCs (WT1-post pulsed) or HPL-IFN-DCs (WT1-pre-pulsed).

### EMBODIMENTS OF THE INVENTION

Hereafter, the present invention is described in detail.

The expression “A to B” (A and B are numerical values) used herein indicates a range of “not less than A to not more than B” unless otherwise specified. The symbol “%” used herein indicates “v/v %” unless otherwise specified.

The present invention relates to a method for separating monocytes from mononuclear cells and a method for preparing dendritic cells (DCs) from monocytes.

Mononuclear cells are leucocytes, and mononuclear cells are classified into monocytes and lymphocytes. Mononuclear cells encompass peripheral blood-derived mononuclear cells (PBMCs), bone marrow-derived mononuclear cells, spleen cell-derived mononuclear cells, and umbilical blood-derived mononuclear cells. PBMCs are particularly preferable. Mononuclear cells can be separated with the use of an apparatus for blood component sampling (apheresis). Unfrozen fresh mononuclear cells or frozen mononuclear cells may be used. Even when frozen mononuclear cells are used, cytotoxic activity of the dendritic cells as the final products would not be lowered.

In the method for preparing dendritic cells from monocytes according to the present invention, monocytes separated by the method for separating monocytes from mononuclear cells according to the present invention or monocytes separated by other methods may be used. Monocytes encompass peripheral blood-derived monocytes, bone marrow-derived monocytes, spleen cell-derived monocytes, and umbilical blood-derived monocytes. Peripheral blood-derived monocytes are particularly preferable. Monocytes are positive for CD14. When monocytes are collected from an organism, monocytes can be separated on the basis of the presence of CD14 as the indicator with the use of a fluorescent activated cell sorter (FACS), a flow cytometer, a magnetic separator, or the like. Monocytes can be separated with the use of an apparatus for blood component sampling (apheresis). Monocytes can also be separated by density gradient centrifugation using, for example, Ficoll*. Monocytes may be derived from any animal species without particular limitation. Examples of such animals include mammalian animals, such as mice, rats, guinea pigs, hamsters, rabbits, cats, dogs, sheep, pigs, cows, horses, goats, monkeys, and humans. Examples of FACS and a flow cytometer that can be used include FACS vantage (Becton, Dickinson and Company) and FACS Calibur (Becton, Dickinson and Company). An example of a magnetic separator that can be used is autoMACS® (Miltenyi Biotec). For example, monocytes can be separated from peripheral mononuclear cells (PBMCs) on the basis of CD14 expression as the indicator using CD14 microbeads comprising CD14 bound thereto by AutoMACS® and CliniMACS® technologies.

1. Separation of Monocytes from Mononuclear Cells

In the method for separating monocytes from mononuclear cells according to the present invention, mononuclear cells are seeded in an adhesion culture dish and cultured therein, and monocytes are allowed to adhere to the culture dish. Thus, monocytes are separated from lymphocytes.

In this case, a medium that is supplemented with a platelet lysate (PL) but is not supplemented with the serum (i.e., a serum-free medium) is used as a culture solution. A human platelet lysate (HPL) derived from the human platelet is preferably used. HPL is a purified human platelet lysate and it can be purified from the platelet in the plasma. HPL includes platelet-derived growth factors, such as PDGF, TGF-β, IGF-1, and EGF.

A method for preparing HPL is not limited. For example, HPL can be obtained by subjecting the platelet to freeze-thawing. Specifically, the platelet (1.5×10⁹/ml) in the plasma may be frozen at −80° C. and thawed to lyse the platelet. HPL prepared by pooling the platelets obtained from many blood donors is also preferable. Commercially available HPL can be used. For example, UltraGRO™-PURE and UltraGROT™-PURE GI (AventaCell BioMedical) can be used. Concerning HPL, an extent of lot-to-lot variation of the same manufacturer is small, and an extent of variation among manufacturers is also small.

Mononuclear cells can be cultured in vitro in accordance with a well-known human lymphoid cell culture technique.

A serum-free medium to be supplemented with HPL is not limited, and a medium that can be used for human lymphoid cell culture can be used. For example, DCO-K (Nissui Pharmaceutical Co., Ltd.), AIM-V™ (Thermo Fisher Scientific), X-VIVO5™, HL-1™ (Lonza K.K), BIOTARGET™-1 SFM (Cosmo Bio), DMEM, MEM, RPMI 1640, and IMDM can be used. DCO-K (Nissui Pharmaceutical Co., Ltd.) is particularly preferable.

The aforementioned serum-free media may be supplemented with HPL as described above in an amount of 1 to 10 (v/v) %, preferably 2 to 7.5 (v/v) %, more preferably 2.2 to 5.3 (v/v) %, and particularly preferably 2.5 to 5 (v/v) % and used. Concerning HPL, as described above, an extent of lot-to-lot variation of the same manufacturer is small, and an extent of variation among manufacturers is also small. Thus, the same effects can be attained with the use of HPL at the concentration described above, regardless of the manufacturer or production lot.

Monocytes adhere firmly to a vessel. Accordingly, mononuclear cells are subjected to adhesion culture, monocytes are allowed to adhere to a culture vessel, such as a culture dish, a petri dish, a plate, or a flask, and cells that do not adhere to a culture vessel are removed. Thus, monocytes can be separated and collected. A vessel for adhesion cell culture to which cells can adhere may be used. A wide variety of commercially available vessels for adhesion cell culture can be used. As a vessel for adhesion cell culture, a low-adhesion culture vessel or a high-adhesion culture vessel may be used.

At the time of culture, a pH level is preferably about 6 to 8. Culture may be performed generally at about 30° C. to 40° C. for 15 minutes to 12 hours, preferably for 15 minutes to 6 hours, more preferably for 15 minutes to 3 hours, more preferably for 15 minutes to 1 hour, more preferably for 20 minutes to 45 minutes, and particularly preferably for 25 minutes to 35 minutes. When a culture period exceeds a day, cells are suspended and detached. At the time of culture, medium exchange, aeration, and agitation may be additionally performed, according to need. For example, carbon dioxide may be added, and carbon dioxide may be added in an amount of 2.5 to 10%, preferably 2.5 to 7.5%, and more preferably 5%.

After adhesion culture, cells that did not adhere to the vessel are removed by washing, and monocytes can be separated as adherent cells. In this case, washing is performed 1 to 5 times, and preferably 2 times.

2. A Method for Preparing Dendritic Cells (DCs) from Monocytes

With the use of the monocytes separated by the method for separating monocytes from mononuclear cells, dendritic cells can be prepared. The separated monocytes are cultured by non-adhesion culture; i.e., suspension culture. Non-adhesion culture may be performed with the use of a culture vessel, such as a non-adherent plate, dish, or flask. A non-adherent culture vessel comprises a culture dish having a surface, which is coated with a compound, such as an ultrahydrophilic polymer, a phospholipid polymer, or an MPC polymer, hydrophilized without the use of a coating agent, or prepared to prevent cells from adhering thereto. For example, low-adhesion culture dishes, such as HydroCell™ (CellSeed), EZ-BindShut® II (Iwaki), Nunclon™ Vita, and Lipidure® coat (NOF Corporation), can be used.

At the outset, the separated monocytes are induced to differentiate into DCs. As a result, immature DCs are obtained. Subsequently, immature DCs are cultured in the presence of particular cytokines for maturation. Thus, mature DCs having cytotoxic activity can be obtained.

Monocytes may be induced to differentiate into DCs by culture in a serum-free medium containing cytokines having activity of inducing DC differentiation and HPL. The serum-free medium described with respect to the method for separating monocytes from mononuclear cells above can be used, and DCO-K (Nissui Pharmaceutical Co., Ltd.) is particularly preferable. Also, HPL described with respect to the method for separating monocytes from mononuclear cells above can be used, and the concentration thereof to be added is as described with respect to the method for separating monocytes from the mononuclear cells.

As cytokines having activity of inducing DC differentiation, the granulocyte macrophage colony-stimulating factor (GM-CSF) and IFN-α may be used. IFN-α is preferably PEGylated-interferon-α (PEG-IFN-α).

PEG-IFN-α comprises polyethylene glycol (PEG) bound to IFN-α. PEG-IFN-α is preferably PEG-IFN-α-2b. As PEG-IFN-α, a commercially available PEG-IFN preparation can be used. An example of a commercially available PEG-IFN-α preparations is a PEG-IFN-α-2b preparation; i.e., PEG-Intron (PEGINTRON®) (common name: Peginterferon Alfa-2b (Genetic recombination)).

PEG-Intron® is represented by a structural formula: H₃C—(O—CH₂CH₂)n-OCO-Interferon alfa-2b, which is composed of a mole of methoxy polyethylene glycol (average molecular weight: about 12,000) covalently bound to a site of amino acid residues (Cys 1, His 7, Lys 31, His 34, Lys 49, Lys 83, Lys 112, Lys 121, Tyr 129, Lys 131, Lys 133, Lys 134, Ser 163, and Lys 164) of Interferon α-2b (genetic recombination) (molecular weight: 19268.91) via a carbonyl group. The molecular weight thereof is about 32,000, and the molecular formula thereof is C₈₆0H₁₃₅₃N₂₂₉O₂₅₅S₉. The CAS registry number is 215647-85-1.

When monocytes are used at the concentration of 10 to 10⁷ cells/ml, for example, the concentration of GM-CSF used for culture is 100 U/ml to 10,000 U/ml, preferably 500 U/ml to 2,000 U/ml, more preferably 800 U/ml to 1,200 U/ml, and particularly preferably 1,000 U/ml. Alternatively, such concentration is 10 ng/ml to 1,000 ng/ml, preferably 20 ng/ml to 200 ng/ml, and more preferably 20 ng/ml to 100 ng/ml. The concentration of PEG-IFN-α is 100 ng/ml to 10 μg/ml, preferably 500 ng/ml to 5 μg/ml, and more preferably 500 ng/ml to 2 μg/ml.

In the presence of HPL. GM-CSF, and PEG-IFN-α, culture is performed for 2 to 5 days, preferably for 3 to 4 days, and more preferably for 3 days. As a result of culture performed in the presence of HPL. GM-CSF, and PEG-IFN-α, immature DCs are obtained.

Immature DCs are cultured in a maturation medium and mature DCs are obtained. As a maturation medium, a serum-free medium containing HPL, GM-CSF, PEG-IFN-α, prostaglandin E2 (PGE2), and OK432 is used. GM-CSF, PEG-IFN-α, and prostaglandin E2 are cytokines. The serum-free medium described with respect to the method for separating monocytes from mononuclear cells above can be used, and DCO-K (Nissui Pharmaceutical Co., Ltd.) is particularly preferable. The HPL described with respect to the method for separating monocytes from mononuclear cells above can be used, and the concentration thereof to be added is as described with respect to the method for separating monocytes from mononuclear cells above.

When monocytes are used at the concentration of 10⁴ to 10⁷ cells/ml, for example, the concentration of GM-CSF used for culture is 100 U/ml to 10,000 U/ml, preferably 500 U/ml to 2,000 U/ml, more preferably 800 U/ml to 1,200 U/ml, and particularly preferably 1,000 U/ml. Alternatively, such concentration is 10 ng/ml to 1,000 ng/ml, preferably 20 ng/ml to 200 ng/ml, and more preferably 20 ng/ml to 100 ng/ml. The concentration of PEG-IFN-α is 100 ng/ml to 10 μg/ml, preferably 500 ng/ml to 5 μg/ml, and more preferably 500 ng/ml to 2 μg/ml. The concentration of PGE2 is 1 ng/ml to 100 ng/ml, preferably 5 ng/ml to 50 ng/ml, and more preferably 5 ng/ml to 20 ng/ml. The concentration of OK432 is 1 μg/ml to 100 μg/ml, preferably 5 μg/ml to 50 μg/ml, and more preferably 5 μg/ml to 20 μg/ml.

The expression of the monocyte or DC surface antigen may be inspected by FACS or other means, so as to determine the adequate concentration at which cells with the target extent of differentiation can be obtained.

Culture may be performed in a maturation medium for 10 to 48 hours, preferably for 10 to 36 hours, more preferably for 10 to 24 hours, and particularly preferably for 18 to 24 hours. Thus, cytotoxic DCs can be obtained.

A total culture period that is necessary to separate monocytes from mononuclear cells and obtain mature cells is for 3 to 7 days, preferably for 4 to 6 days, more preferably for 4 to 5 days, and particularly preferably for 4 days.

DCs prepared by the method according to the present invention comprising performing culture in a serum-free medium containing HPL and cytokines, such as IFN, are referred to as “HPL-IFN-DCs.” In contrast, DCs prepared by the method comprising performing culture m a serum-free medium not containing HPL, which is different from the serum-free medium used for preparation of HPL-IFN-DC only in this respect; i.e., an HPL-free and serum-free medium, are referred to as “IFN-DCs.”

### 3. Properties of the HPL-IFN-DCs Obtained

### (1) Cell Viability and Yield

In the method of the present invention, DCs are prepared from monocytes via non-adhesion culture. Thus, the viability of DCs is high and the yield is also high. The viability of the DCs obtained is 70% or higher, preferably 80% or higher, more preferably 90% or higher, more preferably 95% or higher, and more preferably 97% or higher, which is the standard level defined by the National Institutes of Health (NIH). The rate of DC collection (i.e., the proportion of the number of viable DCs relative to the number of monocytes seeded) is 5% or higher, preferably 10% or higher, more preferably 15% or higher, and particularly preferably 20% or higher. The purity of DCs is 90% or higher, and preferably 95% or higher. The viability, the yield, and the purity of HPL-IFN-DCs are higher than those of IFN-DCs.

### (2) Surface Antigen

HPL-IFN-DCs have morphological properties such that they have dendritic processes. In addition, HPL-IFN-DCs are found to be positive for surface antigens, such as CD14, CD16, CD56, CD83, CD86, CCR7 (CD197), HLA-ABC, and HLA-DR, as a result of, for example, FACS analysis. CD14 is a monocyte marker, CD56 is a cell adhesion molecule, CD197 (CCR7) is a molecule that promotes migration to the lymph node, and CD11c is a dendritic cell marker. CD80 and CD40 are costimulatory molecules involved in the antigen-presenting ability to T cells, CD83 is a dendritic cell maturation marker, and HLA-DR is a molecule involved in antigen presentation.

Whether the cells of interest are positive or negative for such surface antigens can be determined by, for example, inspecting, by microscopic observation, as to whether or not the cells are stained with the use of chromogenic-enzyme- or fluorescence-labeled antibodies that would react with the antigens of interest. For example, the cells may be immunostained using such antibodies to determine the presence or absence of the surface antigens. Alternatively, the presence or absence of the surface antigens can be determined using magnetic beads comprising such antibodies bound thereto. Alternatively, the presence or absence of the surface antigens can be determined by FACS or flowcytometry. When the cells are negative for the surface antigen, the cells would not be sorted as positive cells by FACS analysis, and expression thereof would not be detected by immunostaining. When the surface antigens are expressed at levels that are undetectable by the techniques described above, the cells of interest are determined as negative cells.

As a result of comparison of the surface antigen expression levels between HPL-IFN-DCs and IFN-DCs, the expression levels of CD14, CD56, CCR7 (CD197), and CD11c on HPL-IFN-DCs are higher than those on IFN-DCs. When the proportion of surface antigen expression (positive cells (%)) in a cell population is determined by flow cytometry, the proportion of CD14-positive IFN-DCs is 60% or lower (the median: 35.8%), and the proportion of CD14-positive HPL-IFN-DCs is 50% or higher (the median: 83.6%). The proportion of CD56-positive IFN-DCs is 60% or lower (the median: 37.6%), and the proportion of CD56-positive HPL-IFN-DCs is 50% or higher (the median: 68.4%). The proportion of CCR7 (CD197)-positive IFN-DCs is 30% or lower (the median: 10.3%), and the proportion of CCR7 (CD197)-positive HPL-IFN-DCs is 20% or higher (the median: 37.8%).

Specifically, the proportion (%) of CD14-positive HPL-IFN-DCs is 1.5 to 2.5 times that of CD14-positive IFN-DCs (positive cells (%)), the proportion (%) of CD56-positive HPL-IFN-DCs is 1.5 to 2.5 times that of CD56-positive IFN-DCs, and the proportion (%) of CCR7 (CD197)-positive HPL-IFN-DCs is 2.5 to 5 times, and preferably 3 to 5 times that of CCR7 (CD197)-positive IFN-DCs.

In contrast, the CD80, CD83, CD40, and HLA-DR expression levels on HPL-IFN-DCs are lower than those on IFN-DCs. When the proportion of surface antigen expression (positive cells (%)) in a cell population is determined by flow cytometry, the proportion of CD80-positive IFN-DCs is 15% or higher (the median: 84.0%), and the proportion of CD80-positive HPL-IFN-DCs is 60% or lower (the median: 33.1%). The proportion of CD83-positive IFN-DCs is 60% or higher (the median: 86.8%), and the proportion of CD83-positive HPL-IFN-DCs is 80% or lower (the median: 64.2%). The proportion of CD40-positive IFN-DCs is 55% or higher (the median: 98.6%), and the proportion of CD40-positive HPL-IFN-DCs is 95% or lower (the median: 66.9%). The proportion of HLA-DR-positive IFN-DCs is 95% or higher (the median: 99.8%), and the proportion of HLA-DR-positive HPL-IFN-DCs is lower than 100% (the median: 92.7%).

Specifically, the proportion (%) of CD80-positive HPL-IFN-DCs is 0.3 to 0.5 times that of CD80-positive IFN-DCs, the proportion (%) of CD83-positive HPL-IFN-DCs is 0.6 to 0.9 times that of CD83-positive IFN-DCs, the proportion (%) of CD40-positive HPL-IFN-DCs is 0.5 to 0.8 times that of CD40-positive IFN-DCs, and the proportion (%) of HLA-DR-positive HPL-IFN-DCs is 0.8 to 0.95 times that of HLA-DR-positive IFN-DCs.

### (3) Antigen Phagocytic Ability and Degradation Ability

In HPL-IFN-DCs, the antigen phagocytic ability and the antigen degradation ability are improved, compared with those in IFN-DCs. For example, 100 ng/ml FITC-dextran (Molecular Probes, Eugene, OR, U.S.A.) and 10 μg/ml DQ-ovalbumin (Molecular Probes) are added to a maturation medium, culture is performed for 24 hours, the collected IFN-DCs or HPL-IFN-DCs are washed 2 times with PBS, the resulting cells are resuspended in 1 (v/v) % FBS-PBS, and the phagocytic ability and the degradation ability are then evaluated by flow cytometry. Thus, the results as described below are obtained. While FITC-dextran ΔMFI (the antigen phagocytic ability) of IFN-DCs is 30 or lower (the mean: 17.1), that of HPL-IFN-DCs is 50 or higher (the mean: 68). While DQ-ovalbumin ΔMFI (the antigen degradation ability) of IFN-DCs is 450 or lower (the mean: 270.9), that of HPL-IFN-DCs is 350 or higher (the mean: 589.7).

Specifically, FITC-dextran ΔMFI (the antigen phagocytic ability) of HPL-IFN-DCs is 2 to 6 times, and preferably 3 to 5 times that of IFN-DCs, and DQ-ovalbumin ΔMFI (the antigen degradation ability) of HPL-IFN-DCs is 1.5 to 3 times that of IFN-DCs.

### (4) Cytokine-Producing Ability

The amounts of cytokine production indicated below are determined by suspending mature HPL-IFN-DCs to a cell density of 1×10⁶ cells/ml in a DCO-K medium, seeding the cell suspension in a culture dish, performing culture at 37° C. in the presence of 5% CO₂ for 24 hours, collecting a culture supernatant, and measuring the amount of cytokines in the collected culture supernatant using the Bio-plex assay kit (Bio-Rad Labs). The amount of production is an average of a plurality of measurement instances (e.g., n=6).

In HPL-IFN-DCs, the amount of the produced Th1 cytokines, IL-12 (p70), which promotes cytotoxic T cell induction, is significantly lower than that in IFN-DCs. While the average amount of production in IFN-DCs is 1.1 μg/ml, that in HPL-IFN-DCs is 0.18 μg/ml.

The amount of the produced Th2 cytokines, IL-10 and TGF-0, which suppress cytotoxic T cell induction, in HPL-IFN-DCs is higher than that in IFN-DCs. While the average amount of IL-10 production in IFN-DCs is 11.47 μg/ml, that in HPL-IFN-DCs is 132.7 μg/ml. While the average amount of TGF-β production in IFN-DCs is 8.02 μg/ml, that in HPL-IFN-DCs is 9.38 μg/ml.

Specifically, the amount of IL-10 production in HPL-IFN-DCs is 8 to 15 times, and preferably 9 to 13 times that in IFN-DCs, and the amount of TGF-β production in HPL-IFN-DCs is 1.1 to 1.5 times that in IFN-DCs.

In addition, the amount of production of TNF-α and IL-6 involved in induction of inflammation to activate and differentiate T cells in HPL-IFN-DCs is higher than that in IFN-DCs. While the average amount of TNF-α production in IFN-DCs is 412.5 μg/ml, that in HPL-IFN-DCs is 1144.4 μg/ml. While the average amount of IL-6 production in IFN-DCs is 302.3 μg/ml, that in HPL-IFN-DCs is 2883 μg/ml.

Specifically, the amount of TNF-α production in HPL-IFN-DCs is 2 to 4 times that in IFN-DCs, and the amount of IL-6 production in HPL-IFN-DCs is 8 to 15 times, and preferably 8 to 13 times that in IFN-DCs.

Accordingly, the amount of Th1/Th2 cytokines decreases because of the presence of HPL in a medium used for differentiation and maturation of DCs.

### (5) Cytotoxic T Cell-Inducing Ability

In HPL-IFN-DCs, the cytotoxic T cell-inducing ability is enhanced, compared with that in IFN-DCs.

### (6) Antigen-Specific IFN-γ-Producing Ability by Induced Cytotoxic T Cells

In HPL-IFN-DCs, the antigen-specific IFN-γ-producing ability by the induced cytotoxic T cells is enhanced, compared with that in IFN-DCs.

### 4. A Method of Dendritic Cell Therapy

DCs prepared by the method of the present invention can be used in a method of dendritic cell therapy. An example of a method of dendritic cell therapy is a method of cancer immunotherapy that is known as a method of dendritic cell vaccine therapy. For example, dendritic cells are prepared from monocytes of a subject by the method of the present invention, and the resulting dendritic cells are returned to the subject. Thus, dendritic cells can be used for treatment and prevention of cancer. In such a case, the dendritic cells prepared can act regardless of a cancer type and exert therapeutic effects on cancer. When preparing dendritic cells, a cancer-specific antigen that is specific to a particular type of cancer may be added and cultured. Thus, a cancer-specific antigen is incorporated into dendritic cells, and dendritic cells having cancer-type-specific antitumor immune activity can be obtained. A process of culture performed with the addition of a cancer-specific antigen that is specific to a particular type of cancer at the time of dendritic cell preparation is referred to as pulsing of dendritic cells with a cancer-specific antigen. Pulsing may be performed with the addition of a cancer-specific antigen when preparing cytotoxic dendritic cells from monocytes. Alternatively, pulsing may be performed by first preparing cytotoxic dendritic cells from monocytes, and then subjecting dendritic cells to culture with a cancer-specific antigen. The former is referred to as “pre-pulsing,” and the latter is referred to as “post-pulsing.” A procedure of obtaining dendritic cells having cancer-type-specific antitumor immune activity is referred to as induction of cancer-antigen cytotoxic dendritic cells. Examples of cancer-specific antigens include WT1 peptides in leukemia and other cancers, HER2/neu in breast cancer, CEA (carcinoembryonic antigen) in large bowel cancer, MART-1 (melan-a protein) and MEGA (melanoma antigen) in melanoma, GPC3 (glypican 3) in hepatic cell carcinoma, and PAP (prostate acid phosphatase) and PSMA (prostate specific membrane antigen) in prostate cancer. In the present invention, the dendritic cells can induce cancer-type-specific cytotoxic T cells (CTLs). Dendritic cells having cancer-type-specific antitumor immune activity can be used for treatment of, for example, lung cancer, gastric cancer, pancreatic cancer, liver cancer, rectal cancer, colon cancer, breast cancer, esophageal cancer, uterine cancer, renal cancer, bladder cancer, lymphoma/leukemia, brain tumor, urethral cancer, renal pelvic and ureteral cancer, and mesoepithelioma.

The growth of cancer antigen-specific CTL in a subject can be inspected by the tetramer method or the Elispot assay method.

The present invention encompasses a method for preparing cancer-antigen-specific cytotoxic dendritic cells from monocytes comprising subjecting monocytes separated from the peripheral blood to non-adhesion culture using a serum-free medium containing a human platelet lysate (HPL), GM-CSF, and PEGylated interferon α, adding prostaglandin E2 and OK432, and performing further non-adhesion culture in which, when adding prostaglandin E2 and OK432, a cancer-specific antigen is further added. In such method, for example, non-adhesion culture may be performed for 2 to 5 days with the use of a serum-free medium containing a human platelet lysate (HPL), GM-CSF, and PEGylated interferon α, and culture may be performed for an additional 1 to 2 days with the addition of prostaglandin E2, OK432, and a cancer-specific antigen. While the concentration of the cancer-specific antigen is not limited, the concentration is 0.1 to 1000 μg/ml, preferably 1 to 500 μg/ml, and more preferably 5 to 300 g/ml.

The present invention also encompasses cancer-antigen-specific cytotoxic dendritic cells obtained by the method for preparing cancer-antigen-specific cytotoxic dendritic cells from monocytes.

Such dendritic cells can also be used for treatment of bacterial or virus infections. For treatment of infections, DCs prepared by the method of the present invention in which monocytes are subjected to non-adhesion culture in the presence of HPL, GM-CSF, PEG-IFN-α, PGE2, and OK432 are useful. The prepared dendritic cells may be administered to a subject by, for example, the intracutaneous, subcutaneous, intravenous, or lymphatic route. A dose and a timing of administration can be adequately determined in accordance with a disease type of a subject, disease severity, and subject's conditions.

### 5. DC Differentiation and Inducing Agent

The present invention encompasses an agent for differentiation and induction of DCs from monocytes containing HPL, GM-CSF, and PEG-IFN-α (i.e., a DC differentiation and inducing agent). Such DC differentiation and inducing agent can also be referred to as a “DC preparing agent.” The DC differentiation and inducing agent may further contain PGE2 and OK432. The DC differentiation and inducing agent may be composed of a first reagent containing HPL, GM-CSF, and PEG-IFN-α and a second reagent containing PGE2 and OK432. The present invention also encompasses a DC differentiation and inducing kit comprising the first reagent and the second reagent. The first reagent containing HPL, GM-CSF, and PEG-IFN-αis used for differentiation and induction of immature DCs, and the second reagent containing PGE2 and OK432 is used for maturation of immature DCs.

According to the method of the present invention. DCs are induced to be mature DCs. The present invention also encompasses DCs obtained by the method of the present invention and a cell population including such DCs. In such cell population, the content of DCs is 10% or more, 30% or more, 50% or more, 70% or more, 90% or more, or 95% or more.

### EXAMPLES

The present invention is described in greater detail with reference to the following examples, although the present invention is not limited to these examples.

In the examples of the present invention, DCs prepared with the use of a medium supplemented with HPL and IFN are referred to as “HPL-IFN-DCs,” and DCs prepared with the use of a HPL-free medium supplemented with IFN are referred to as “IFN-DCs.”

### [Example 1] Establishment of a Method for Separating Monocytes and a Method for Preparing IFN-DCs Using a Serum-Free Medium (DCO-K) with Optimized Additives (ABS or HPL)

This example was performed as preliminary testing.

This example is aimed at establishing a method for separating monocytes from the peripheral blood mononuclear cells and a method for preparing IFN-DCs using a serum-free (DCO-K) medium (Nissui Pharmaceutical Co., Ltd.) containing additives at the optimized concentration (human serum type AB (ABS) (Biowest) and a human platelet lysate (HPL) (AventaCell Biomedical Co., Ltd.)). While an example in which DCO-K is used as a serum-free medium is described herein, equivalent results can be obtained with the use of other serum-free medium.

Points of evaluation are described below.

(1) IFN-DCs were prepared using a DCO-K medium containing ABS or HPL at the optimized concentration, and cell morphology was observed under a phase contrast microscope (EVOS® FL Cell Imaging System).
(2) The cell viability of IFN-DCs was measured based on the results of staining of dead cells with trypan blue, and the yield and the purity were evaluated by flow cytometry (FCM).
(3) Cells were stained using antibodies reacting with DC markers comprising fluorescent dyes, such as FITC, PE, and APC, added thereto, and the IFN-DC phenotype was examined by flow cytometry.

In general, dendritic cell (DC) vaccines are prepared by adhesion culture in which monocytes as starting materials are separated from the peripheral blood mononuclear cells (PBMCs, which include monocytes and lymphocytes). Monocytes strongly adhere to a dish. PBMCs collected from a patient via apheresis were suspended in a serum-free medium (DCO-K) supplemented with additives (ABS or HPL at the final concentration of 5 (v/v) %) or an AIM-V medium without additives (a conventional method, the AIM-V medium), and the suspension was seeded in an adhesion culture dish. Culture was performed at 37° C. in the presence of 5% CO₂ for 24 hours or 30 minutes to allow the cells to adhere to the bottom of the culture dish, and monocytes (starting materials of IFN-DC vaccines) were separated from lymphocytes. Subsequently, adherent cells were induced to differentiate into IFN-DCs using a DCO-K medium supplemented with 1 μg/ml PEG-Intron, 100 ng/ml GM-CSF, and HPL (final concentration: 5 (v/v) %) or an AIM-V medium. The cells were collected 3 days after the initiation of differentiation, and culture was performed using a maturation medium containing various reagents (1 μg/ml PEG-Intron, 100 ng/ml GM-CSF, 10 μg/ml OK432, and 10 ng/ml PGE2) mixed in a low-adhesion culture dish (Prime surface, Sumitomo Bakelite) and 20 μg/ml of tumor antigen peptides (WT1: Wilms tumor 1) for 18 to 24 hours to allow IFN-DC to mature. IFN-DCs prepared under various conditions were subjected to Preliminary Tests 1 to 7.

### Preliminary Test 1

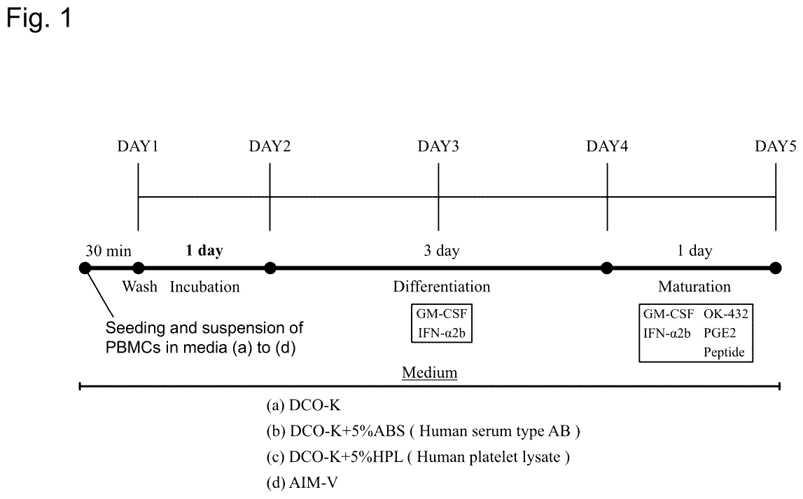

In the process of adhesion culture of peripheral blood mononuclear cells for 24 hours, differentiation, and maturation. IFN-DCs were prepared using a DCO-K medium supplemented with HPL or ABS at the final concentration of 5 (v/v) % or an AIM-V medium alone, and the cell morphology, the cell viability, the purity (DC fractions were defined based on FSC/SSC by flow cytometry and the proportion of DC fractions were calculated as the purity), the contamination rate of lymphocyte, and the phenotypes were compared (n=1). FIG. 1 shows a protocol of Preliminary Test 1.

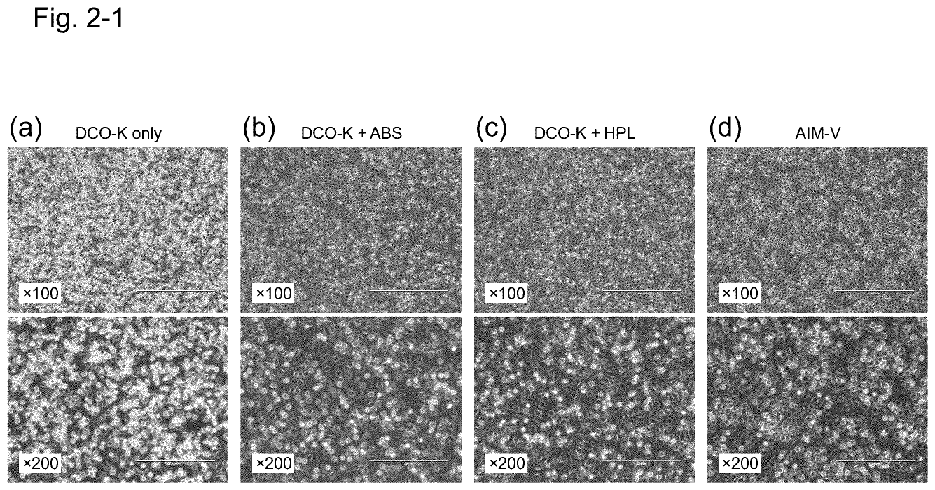

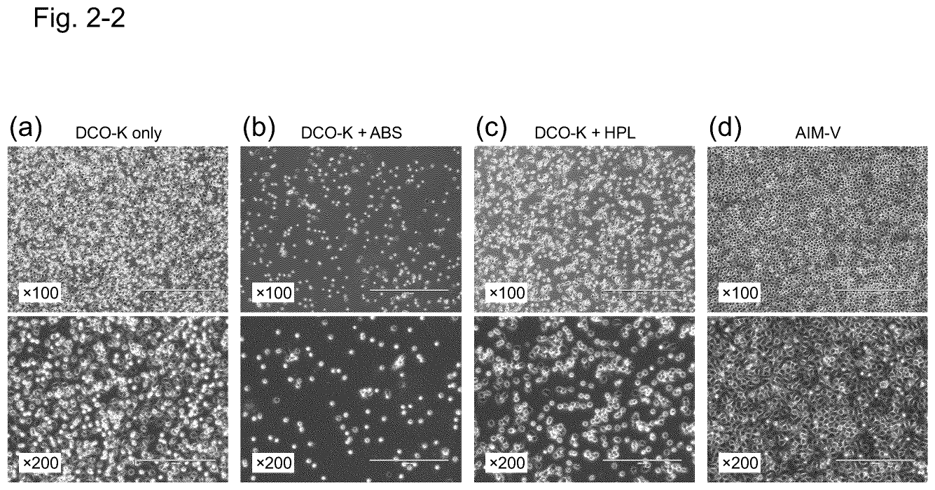

PBMCs were suspended in a serum-free medium (DCO-K) supplemented with additives (ABS or HPL at the final concentration of 5 (v/v) %) or an AIM-V medium alone without additives (a conventional method), the suspension was seeded in an adhesion culture dish (a low-adhesion dish), non-adherent cells were washed 30 minutes later, and cell morphology was observed under a phase contrast microscope (Day 1). FIG. 2 shows cell observation images. (a) shows the results of culture obtained with the use of DCO-K only: (b) shows those obtained with the use of DCO-K+ABS; (c) shows those obtained with the use of DCO-K+HPL: and (d) shows those obtained with the use of AIM-V.

When monocytes in the PBMCs collected from the patient by apheresis are separated from lymphocytes therein in the process of dendritic cell preparation, in general, the peripheral blood mononuclear cells are seeded in a serum-free medium (AIM-V), cells are washed 30 minutes later, adhesion culture is performed for an additional 24 hours, and non-adherent cells are then washed again. Specifically, PBMCs collected from the patient by apheresis were cultured in a serum-free medium (DCO-K) supplemented with ABS or HPL for 24 hours, and cells were observed under a phase contrast microscope (n=1).

PBMCs were suspended in a serum-free medium (DCO-K) supplemented with additives (ABS or HPL at the final concentration of 5 (v/v) %) or an AIM-V medium alone without additives (a conventional method), the suspension was seeded in an adhesion culture dish, non-adherent cells were washed 30 minutes later (Day 1), cells were washed with the medium 24 hours later, and cell morphology was then observed (Day 2). The results are shown in FIG. 2-2. Compared with (a), a larger number of cells were suspended in the DCO-K medium supplemented with ABS or HPL, and cells were detached because of washing. Cells that had been allowed to stand for 1 day after adhesion became suspended. According to a conventional method (d), adherent cells were clearly distinguished from suspended cells as a result of washing.

The cell surface antigens expressed on the IFN-DCs prepared under various conditions were detected using label antibodies by flow cytometry (n=1).

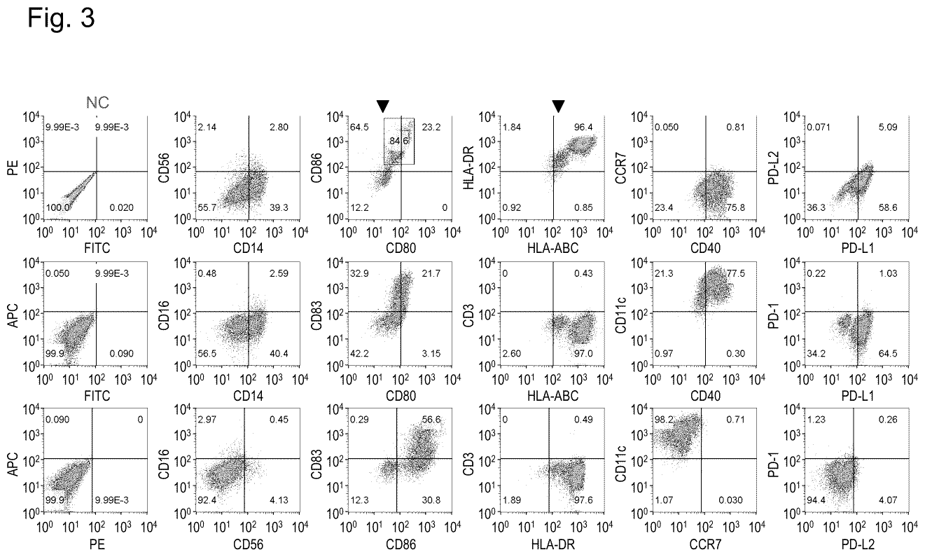

FIG. 3 shows the results concerning the IFN-DCs prepared with the use of the DCO-K medium only. In the IFN-DCs prepared with the use of the DCO-K medium only, expression of costimulatory molecules CD40, CD86, and CD80 involved in the antigen presenting ability to T cells, CD83 as the indicator of dendritic cells maturation, and HLA-DR and HLA-ABC involved in antigen presentation was detected. While immature-like dendritic cells (CD80⁻/CD83⁻/CD86⁻ and HLA-ABC/DR subfractions) were detected, such immature-like phenotypes were considered to result from insufficient maturation reactions affected by cell conditions.

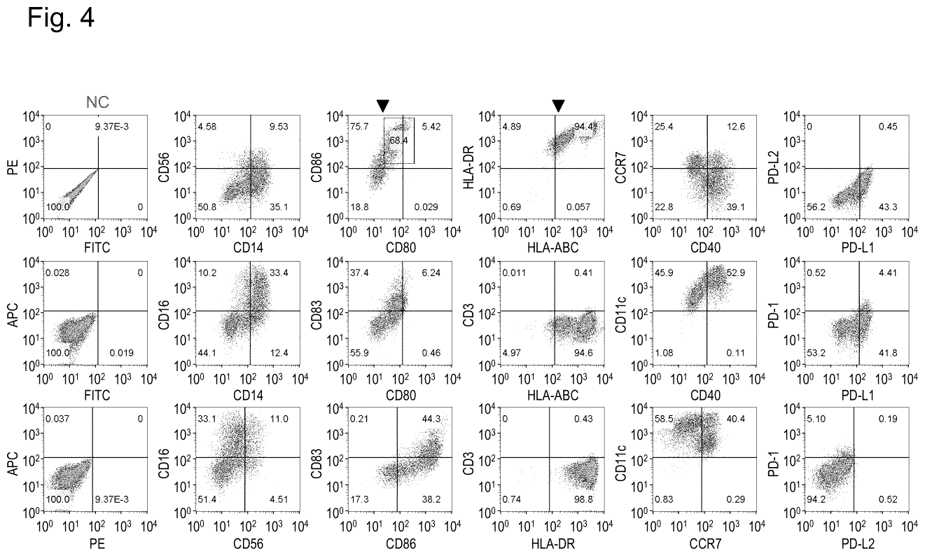

FIG. 4 shows the results concerning IFN-DCs prepared using the DCO-K+ABS medium. When IFN-DCs were prepared using the DCO-K medium supplemented with the serum (ABS) in (b), expression of a phenotype similar to that in (a) was observed, and a larger number of heterogeneous subfractions (CD80⁻/CD83⁻/CD86⁻) than those detected in (a) were observed.

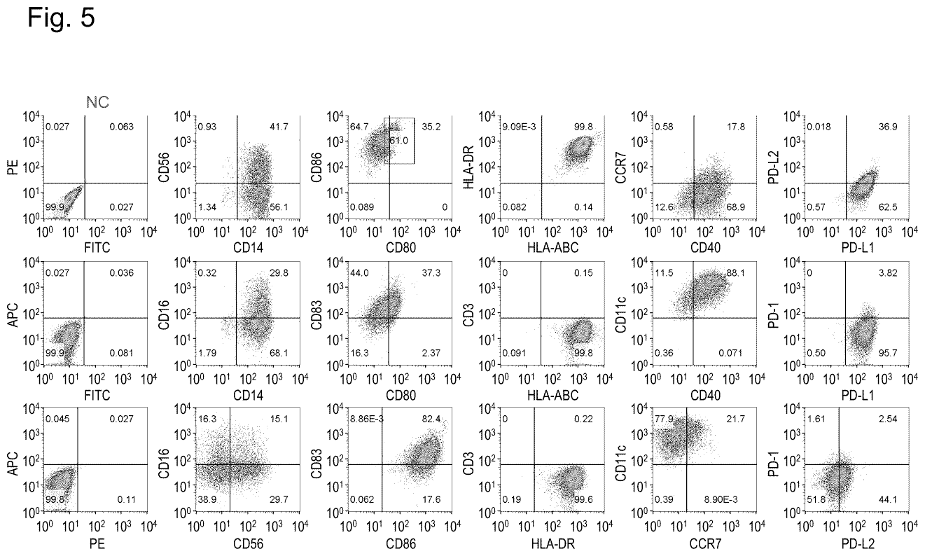

FIG. 5 shows the results concerning IFN-DCs prepared using the DCO-K+HPL medium (HPL-IFN-DC). In comparison with (a) and (b), as well as (d) in which a conventional medium was used, lowered expression of CD80, CD86, and CD83 and expression of CD14, CD16, CCR7, HLA-DR, and HLA-ABC were observed in (c). In particular, expression of CD14, CD16, and CD56 was observed at significant levels (a homogeneous cell population of CD14⁺⁺CD16₊CD56⁺CCR7⁺HLA-ABC⁺HLA-DR⁺) and a phenotype completely different from that of conventional IFN-DC as in the case of (d) was observed.

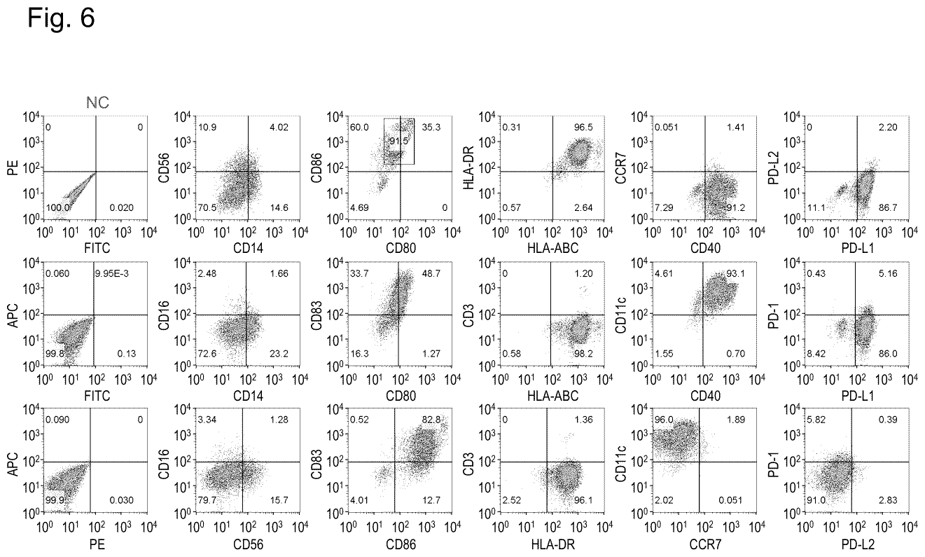

FIG. 6 shows the results concerning IFN-DCs prepared using a conventional AIM-V medium. According to a conventional method, weakly positive expression of CD14 and expression of CD80, CD86, CD83, HLA-ABC, HLA-DR, and CD40 were observed, and such phenotype was found to be similar to that reported in related literature (Terutsugu Koya et. al., Scientific reports 7, Article number 42145: 2017).

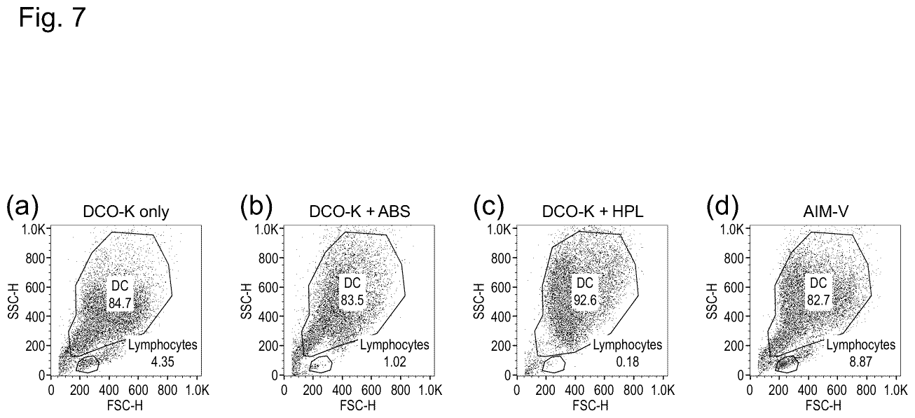

When IFN-DCs prepared under various conditions were collected to prepare DC vaccines, the quality indicators; i.e., the purity and the contamination rate of lymphocyte at the time of IFN-DC collection, were evaluated by flow cytometry. The cells were subjected to an adhesion for 30 minutes, and differentiation induction was initiated immediately thereafter. The results are shown in FIG. 7. While contamination of a large number of lymphocytes was observed in (a) and (d), a lowered rate of lymphocyte contamination was observed in IFN-DCs prepared with the addition of ABS (a) or HPL (c). When HPL was added, in particular, an extent of lowering was significant. High purity was observed in (c). This indicates that lymphocyte-like suspended cells may have been removed by detachment in the process of separation of monocytes from lymphocytes in PBMCs performed on Day 2 with the addition of HPL.

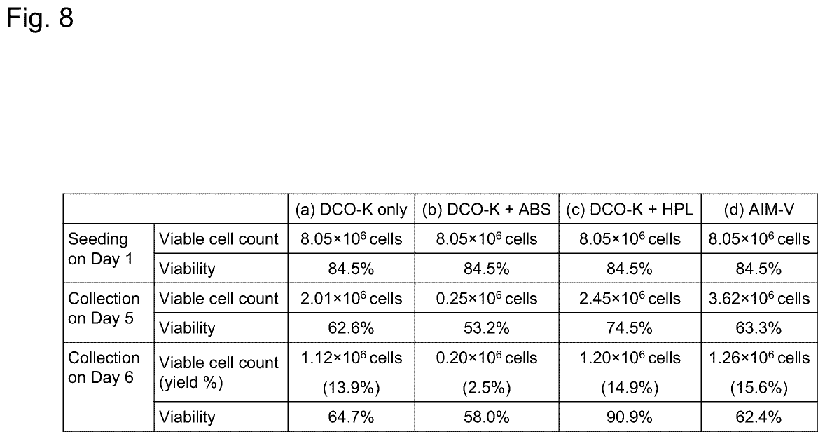

FIG. 8 shows a summary of the cell viability and the yield. The cell viability in the HPL-supplemented serum-free medium DCO-K (a) was very high. The yield in (c) was equivalent to that in (d), the yield in (a) was relatively low, and the yield in (b) was significantly low. The yield (%) is determined by dividing the viable cell count upon collection on Day 6 by the number of viable cells upon seeding on Day 1. The cell viability upon collection was very high in IFN-DCs prepared with the use of the HPL-supplemented DCO-K medium (c).

A summary of Preliminary Test 1 is described below.

As a result of comparison with the conventional method (d), preparation of IFN-DCs with the use of a serum-free medium DCO-K (Nissui Pharmaceutical Co., Ltd.) (a) was found to be feasible. In the IFN-DCs prepared with the use of an HPL-supplemented DCO-K medium (c), the cell viability and the purity were improved, in comparison with other groups ((a), (b), and (d)). In terms of phenotypes, in addition, phenotypes different from those of conventional IFN-DCs, such as CD14⁺⁺, CD16⁺, and CD56⁺, were observed, and a very homogeneous cell population of CD40⁺, CD86⁺, HLA-ABC⁺, and HLA-DR⁺ was formed.

As described above, HPL and DCO-K are suitable for IFN-DC preparation in terms of the cell viability and the purity. In the step of separation of monocytes from PBMCs, however, cells are suspended and detached after they are allowed to stand for 1 day after adhesion. Thus, the yield is deduced to be lowered. Accordingly, a process comprising seeding cells, performing an adhesion for 30 minutes, washing cells 2 times with media, and then inducing differentiation was employed.

### Preliminary Test 2

In the process of adhesion culture of the peripheral blood mononuclear cells for 30 minutes, differentiation, and maturation, IFN-DCs were prepared using the DCO-K medium supplemented with HPL or ABS at the final concentration of 5 (v/v) % or the AIM-V medium without additives, and the cell morphology, the cell viability, the purity, the contamination rate of lymphocyte, and the phenotype were examined (n=1).

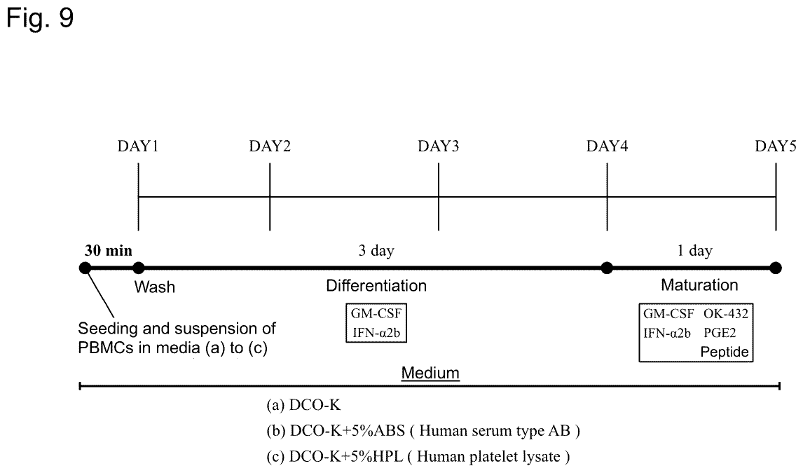

FIG. 9 shows a protocol of Preliminary Test 2.

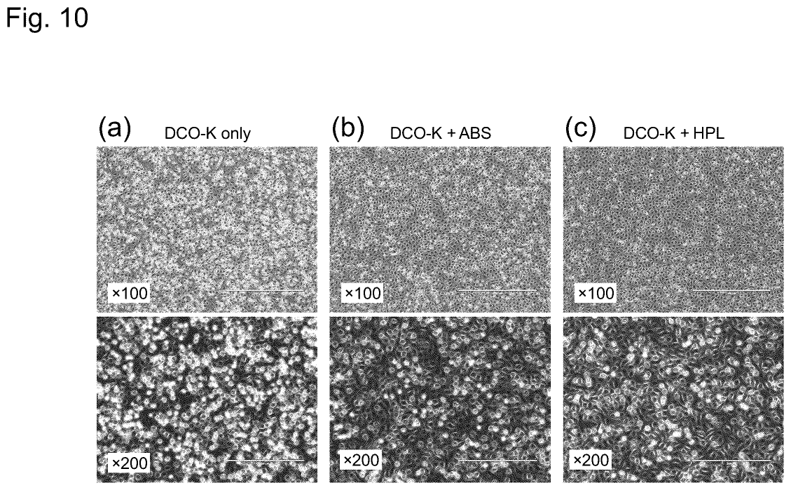

In the step of separating monocytes from lymphocytes in the PBMCs collected by apheresis, adhesion culture was performed for 30 minutes in the DCO-K medium supplemented with ABS at the final concentration of 5 (v/v) % (b) or HPL at the final concentration of 5 (v/v) % (c). Subsequently, cells were washed 2 times with media and observed under a phase contrast microscope (n=1). FIG. 10 shows cell observation images. (a) shows the results attained upon culture in DCO-K only, (b) shows those in DCO-K+ABS, and (c) shows those in DCO-K+HPL.

In the case of (a), a large number of lymphocyte-like cells were contaminated in addition to adherent cells. This indicates that lymphocyte-like cells were not removed by washing. In the DCO-K media supplemented with additives ((b) and (c)), a larger number of cells adhered to the bottom were observed, in comparison with (a). This indicates that a large number of lymphocyte-like cells were removed by washing.

After washing, differentiation induction was initiated with the addition of GM-CSF/IFN-α.

Expression of cell surface antigens on the IFN-DCs prepared under various conditions was evaluated by flow cytometry (n=1).

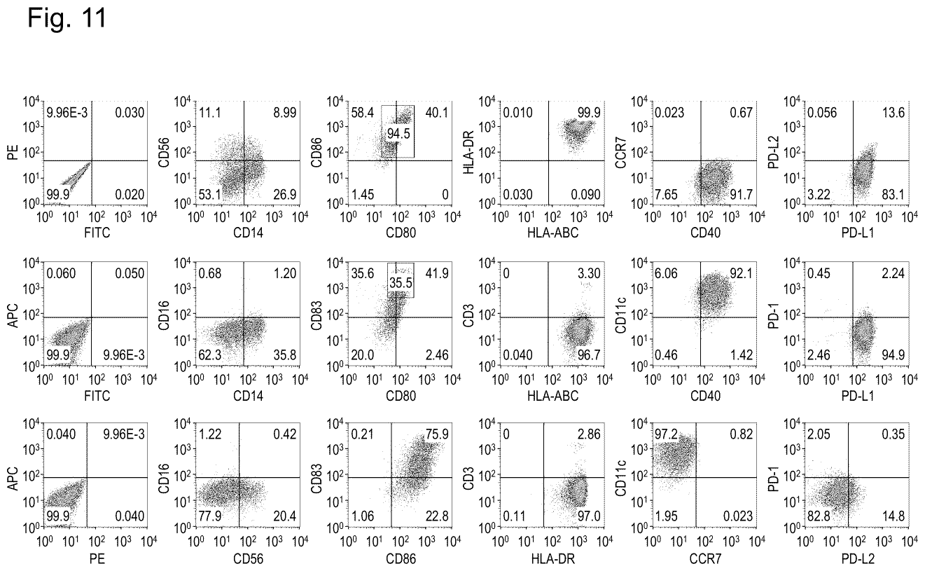

FIG. 11 shows the results obtained upon culture with the use of DCO-K only (a). In comparison with the results obtained in (a) of Preliminary Test 1, a larger number of cells weakly positive for CD14 and positive for CD80, CD86, CD83, HLA-ABC, and HLA-DR were detected, and a phenotype similar to that according to a conventional method ((d) in Preliminary Test 1) was observed.

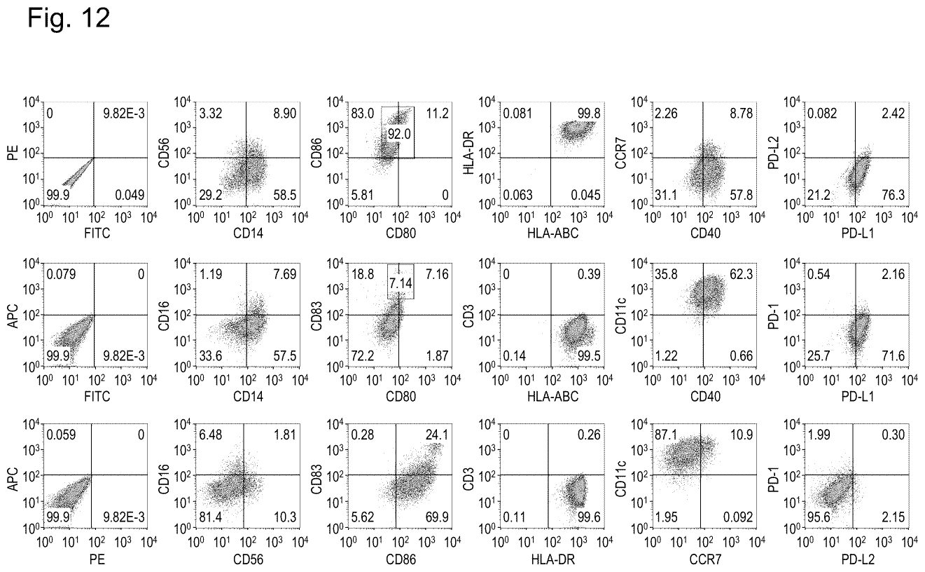

FIG. 12 shows the results attained when cultured in DCO-K+ABS (b). In comparison with (a), elevated expression of CD14 and lowered expression of CD80/CD83 were observed, and a phenotype similar to that of immature-like dendritic cells was observed.

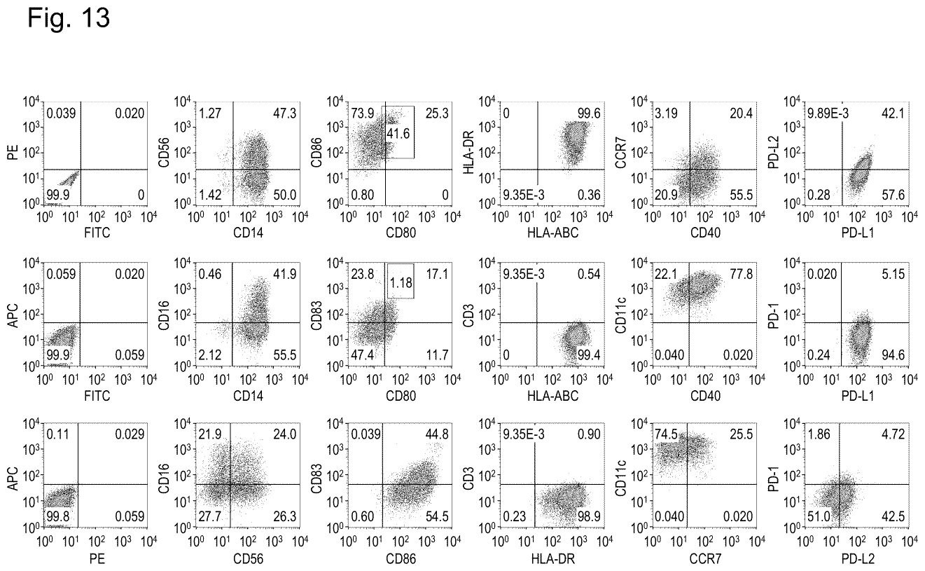

FIG. 13 shows the results attained when cultured in DCO-K+HPL (c). In comparison with other groups ((a) and (b)), lowered expression of CD80/CD83 and a homogeneous cell population of CD14⁺⁺, CD16⁺, CD56⁺, and HLA-ABC/DR⁺ were observed, and a tendency similar to that observed in Preliminary Test 1 was observed.

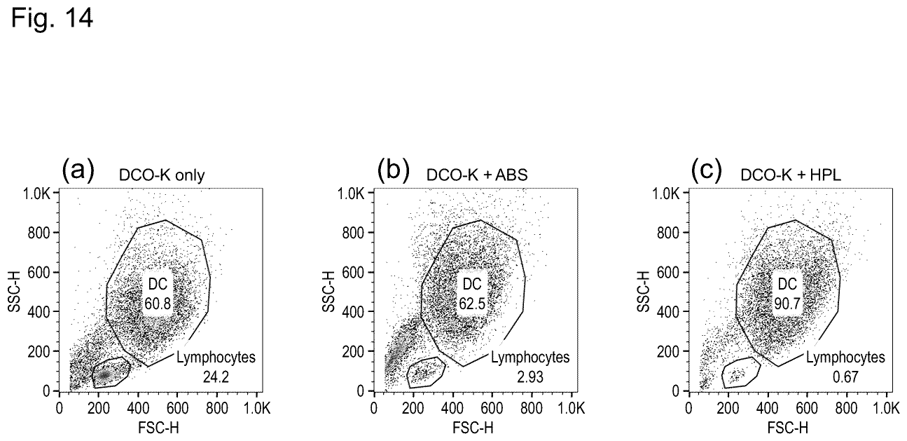

When IFN-DCs prepared under various conditions were collected to prepare DC vaccines, the quality indicators: i.e., the purity and the contamination rate of lymphocyte at the time of IFN-DC collection, were evaluated by flow cytometry. The results are shown in FIG. 14. When a DCO-K medium supplemented with HPL (c) was used in the step of monocyte separation 30 minutes after seeding of PBMCs, the contamination rate of lymphocyte was significantly lower, compared with other groups ((a) and (b)). The contamination rate of lymphocyte was lower than 1%. When differentiation induction was initiated immediately after the adhesion for 30 minutes, contamination of a large number of lymphocytes was observed in (a).

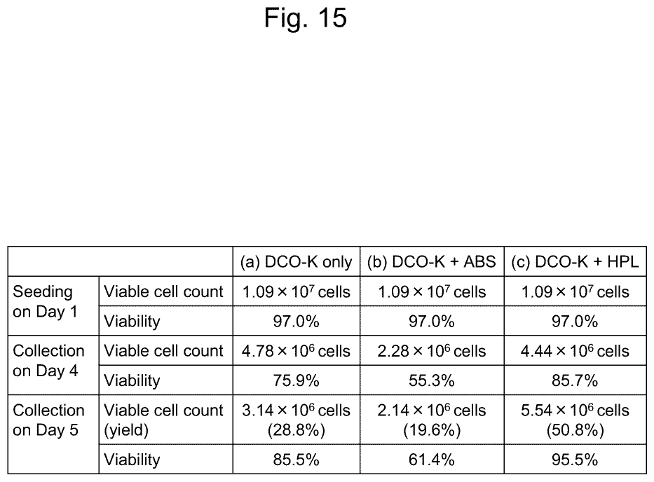

FIG. 15 shows a summary of the cell viability and the yield. The yield (%) is determined by dividing the viable cell count upon collection on Day 5 by the number of viable cells upon seeding on Day 1. The first day of seeding (Day 1) has been shortended to be Day 5. The cell viability was significantly higher in the HHPL-supplemented DCO-K medium (c), in comparison with other groups ((a) and (b)) (n=1).

A summary of Preliminary Test 2 is described below.

In the step of separating monocytes in the PBMCs collected from the patient by apheresis, it is possible to induce IFN-DC differentiation in the adhesion reaction performed 30 minutes after seeding. With the use of the HPL-supplemented DCO-K medium (c) in such a case, the cell viability, the purity, and the rate of collection significantly higher than those observed in other groups ((a) and (b)) were observed. In the HPL-supplemented medium, as with the case in Preliminary Test 1, a homogeneous cell population of CD14⁺⁺, CD16⁺, CD56⁺, CD86⁺, CCR7⁺, HLA-ABC⁺, HLA-DR⁺ surface antigen expression was observed, although the CD80 and CD83 expression levels were low. With the use of the ABS-supplemented DCO-K medium, the cell viability and the yield were lowered, and the expression level of the CD80 fraction was lowered. Thus, the ABS-supplemented DCO-K medium was excluded in the subsequent preliminary tests.

### Preliminary Test 3

In an adhesion culture of peripheral blood mononuclear cells performed for 30 minutes, monocytes were separated from lymphocytes using a DCO-K medium supplemented with HPL at designated concentration (2.5 (v/v) % or 5 (v/v) %). Subsequently, the cell morphology, the cell viability, the purity, the contamination rate of lymphocyte, and the phenotype of IFN-DCs prepared with the DCO-K medium not supplemented with HPL in the process of differentiation and maturation were compared (n=1).

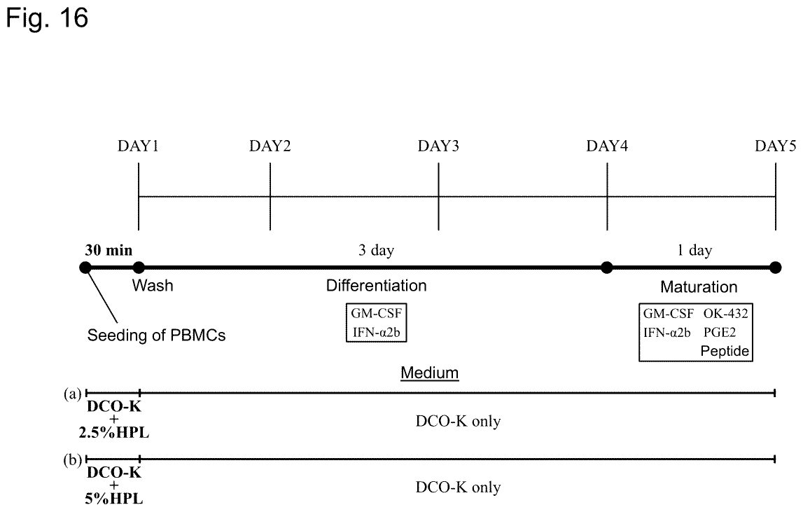

FIG. 16 shows a protocol of Preliminary Test 3.

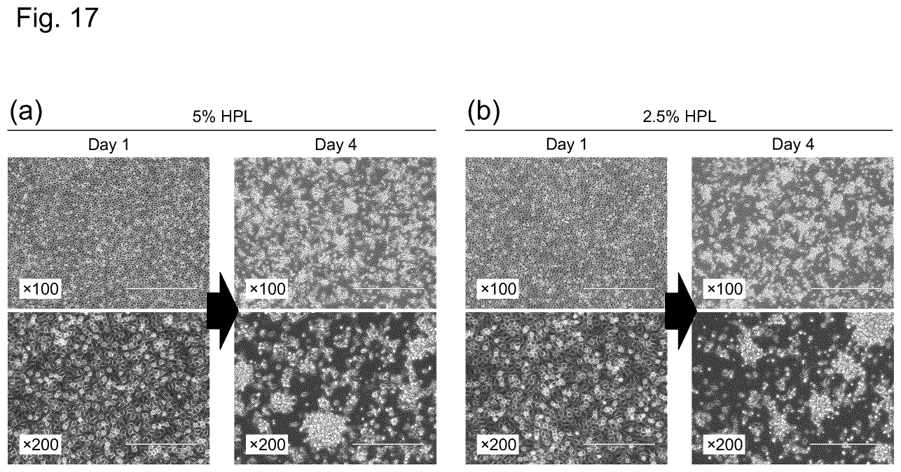

In Preliminary Test 3, no significant differences were detected by cell observation (n=1) when IFN-DCs were prepared with the use of the DCO-K media supplemented with HPL at designated concentrations (2.5 (v/v) % and 5 (v/v) %) only in the step of monocyte separation in a low-adhesion culture dish using PBMCs. FIG. 17 shows cell observation images. (a) shows the results attained when cultured at 2.5 (v/v) % and (b) shows those at 5 (v/v) %.

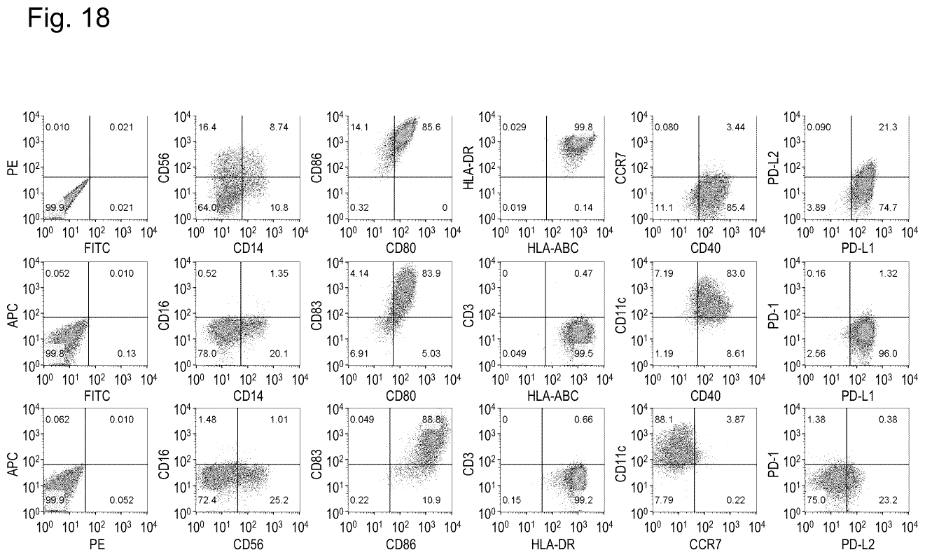

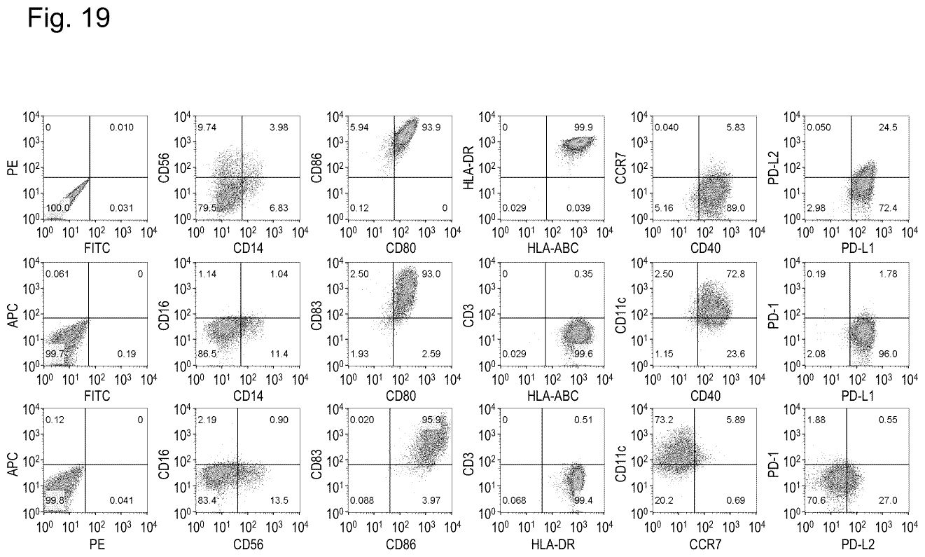

The cell surface antigen expression on IFN-DCs was evaluated by flow cytometry (n=1). FIG. 18 shows the results attained when cultured with HPL at 5 (v/v) % and FIG. 19 shows those with HPL at 2.5 (v/v) %.

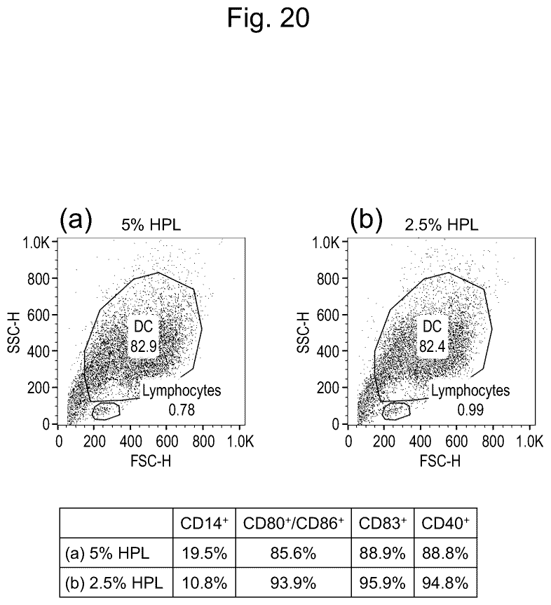

When IFN-DCs prepared under various conditions were collected to prepare DC vaccines, the quality indicators; i.e., the purity and the contamination rate of lymphocyte at the time of IFN-DC collection, were evaluated by flow cytometry. The results are shown in FIG. 20. The IFN-DCs prepared with the use of the DCO-K media supplemented with HPL at designated concentrations (2.5 (v/v) % and 5 (v/v) %) only in the step of monocyte separation in a low-adhesion culture dish using PBMCs exhibited the low contamination rate of lymphocyte (n=1).

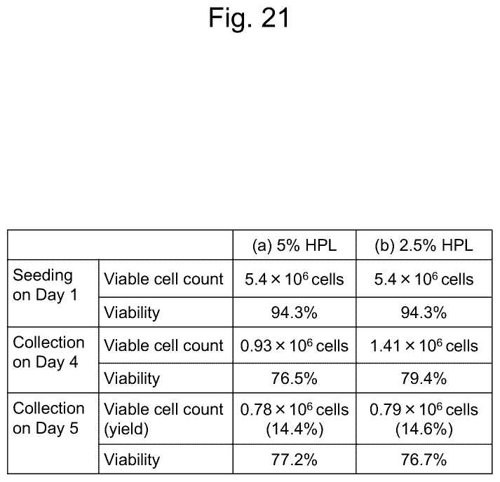

FIG. 21 shows a summary of the cell viability and the yield. The yield (%) is determined by dividing the viable cell count upon collection on Day 5 by the number of viable cells upon seeding on Day 1. The first day of seeding (Day 1) has been shortended to be Day 5. The cell viability was 76% to 77% in the both groups and no significant difference was observed (n=1).

A summary of Preliminary Test 3 is described below.

When HPL was used selectively in the step of monocyte separation in the process of IFN-DC preparation, no significant differences were observed in adhesion properties of the monocytes.

While significant lowering was observed in the contamination rate of lymphocyte in IFN-DCs, the cell viability and the yield were lower than those observed under the conditions in which HPL was added at the time of differentiation and maturation (Preliminary Tests 1 to 2).

The phenotype was similar to that of the IFN-DCs prepared with the use of DCO-K only (Preliminary Tests 1 to 2: (a)). Accordingly, the IFN-DCs prepared with the use of HPL can be expected to show improvement in terms of the cell viability, the yield, and the contamination rate of lymphocyte.

### Preliminary Test 4

In Preliminary Test 4 and subsequent tests, the optimal HPL concentration for IFN-DC preparation was examined.

Preliminary Test 4:

In the process of adhesion culture of the peripheral blood mononuclear cells for 30 minutes, differentiation, and maturation, IFN-DCs were prepared using the DCO-K media supplemented with HPL at designated concentrations (0 to 10 (v/v) %), and the cell morphology, the cell viability, the purity, the contamination rate of lymphocyte, and the phenotype were compared (n=3).

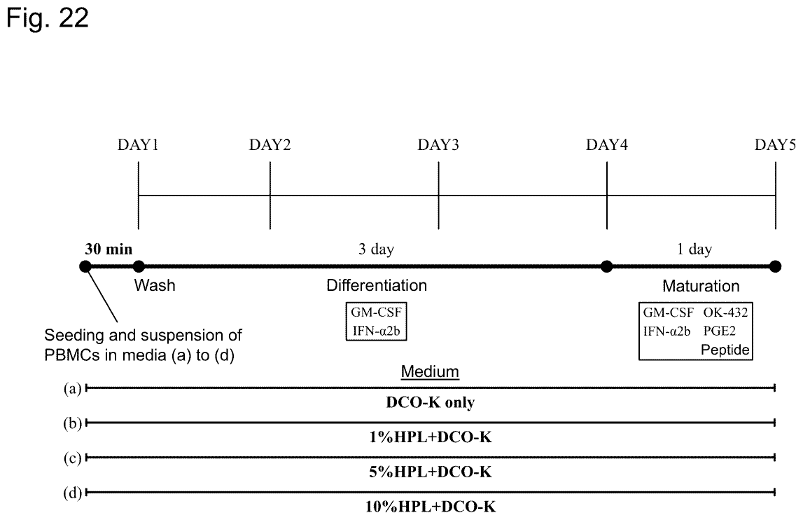

FIG. 22 shows a protocol of Preliminary Test 4.

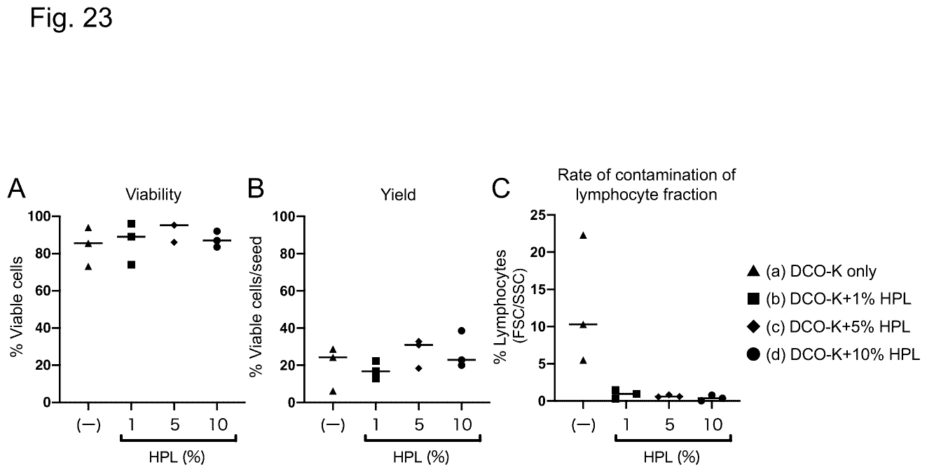

In Preliminary Test 4. IFN-DCs were prepared using the DCO-K media supplemented with HPL at various concentrations (0 (v/v) %, 1 (v/v) %, 5 (v/v) %, and 10 (v/v) %) from the step of monocyte separation to the step of differentiation and maturation, and changes in the cell viability, the yield, the contamination rate of lymphocyte, and the phenotype of IFN-DCs thus prepared were evaluated. The results are shown in FIG. 23. A shows the cell viability, B shows the yield, and C shows the contamination rate of lymphocyte. In comparison with the IFN-DCs prepared with the use of DCO-K only (a), the highest cell viability and the highest yield were observed when HPL was used at 5 (v/v) % (c) (n=3).

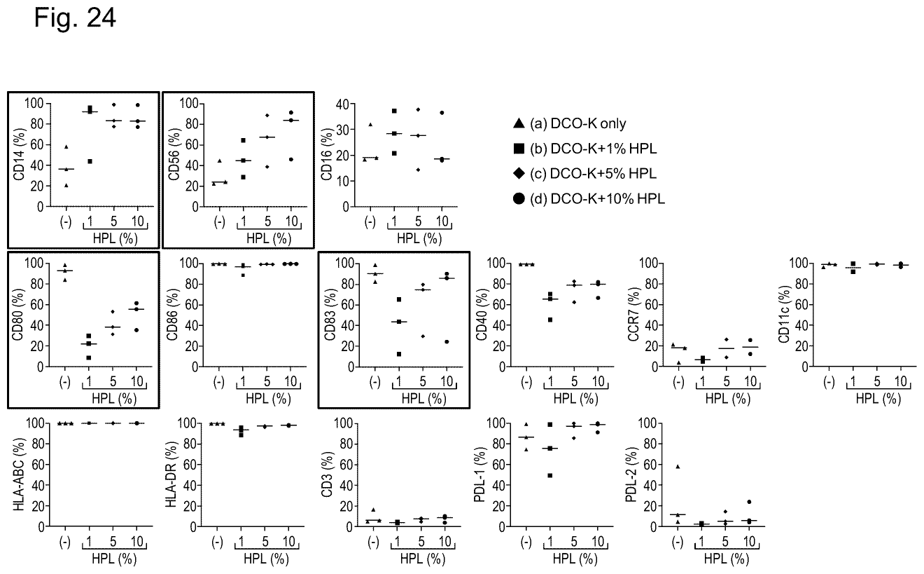

The phenotype of IFN-DCs prepared at various concentrations (0 (v/v) %, 1 (v/v) %, 5 (v/v) %, and 10 (v/v) %) were evaluated by flow cytometry (n=3). The results are shown in FIG. 24.

In comparison with the use of the DCO-K medium only (a), CD14 and CD56 expression levels were elevated in an HPL-concentration-dependent manner, and CD80 and CD83 expression levels were lowered while such expression was recovered in an HPL-concentration-dependent manner. By dot-plotting evaluation, in addition, the convergence of a homogeneous cell population of CD86⁺HLA⁻ABC⁺HLA-DR⁺ was observed in an HPL-concentration-dependent manner.

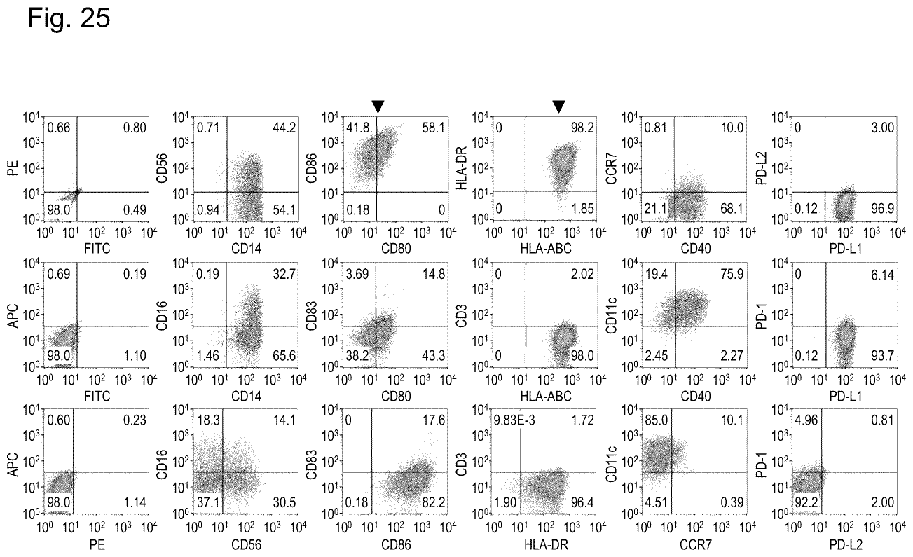

The cell surface antigen expression on IFN-DCs when cultured with HPL at 1 to 10 (v/v) % was evaluated by flow cytometry (n=1). FIG. 25 shows the results obtained when cultured with HPL at 10 (v/v) %. The convergence of a cell population of CD80/CD86 and HLA-ABC/DR was observed in an HPL-concentration-dependent manner.

A summary of Preliminary Test 4 is described below.

IFN-DCs were prepared using the DCO-K media supplemented with HPL at designated concentrations (1 (v/v) %, 5 (v/v) %, and 10 (v/v) %) from the step of monocyte separation to the step of differentiation and maturation, and the cell viability, the yield, the purity, and the phenotype were evaluated by flow cytometry.

In the IFN-DCs prepared with HPL at 1 to 10 (v/v) %, the recovered CD80/CD86 expression levels and the convergence of a cell population of HLA-ABC/DR were observed in a concentration-dependent manner. The IFN-DCs prepared with the further addition of HPL at 5 (v/v) % exhibited the highest cell viability and the highest yield. The results of Preliminary Test 4 demonstrate that the optimal HPL concentration for HPL-IFN-DC is 5 (v/v) % from the viewpoint of the production cost, the cell viability, the yield, and the purity.

### Preliminary Test 5

### Preliminary Test 5:

When preparing IFN-DC preparation with the use of HPL, changes in the phenotype, the cell viability, the yield, and the purity caused by the presence or absence of reagents (HPL, OK432, and cytokines) added in the process of maturation were compared and examined (n=1).

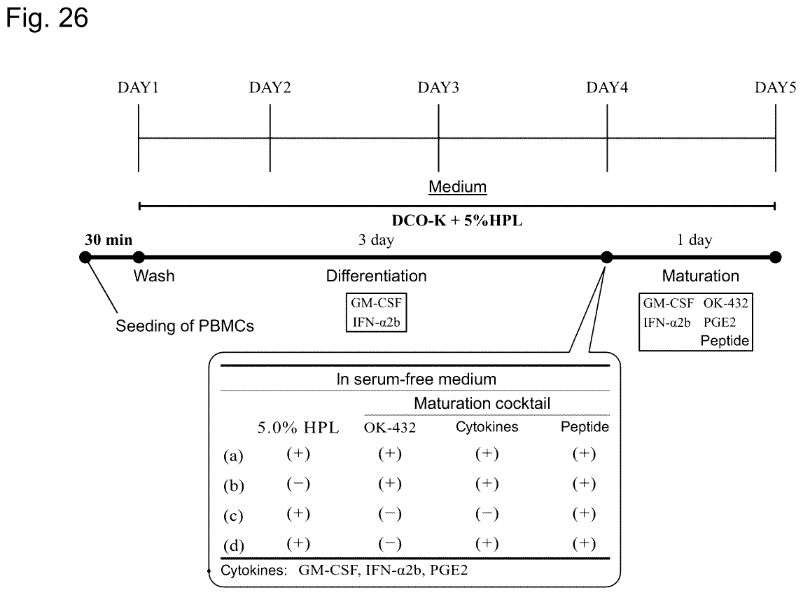

FIG. 26 shows a protocol of Preliminary Test 5.

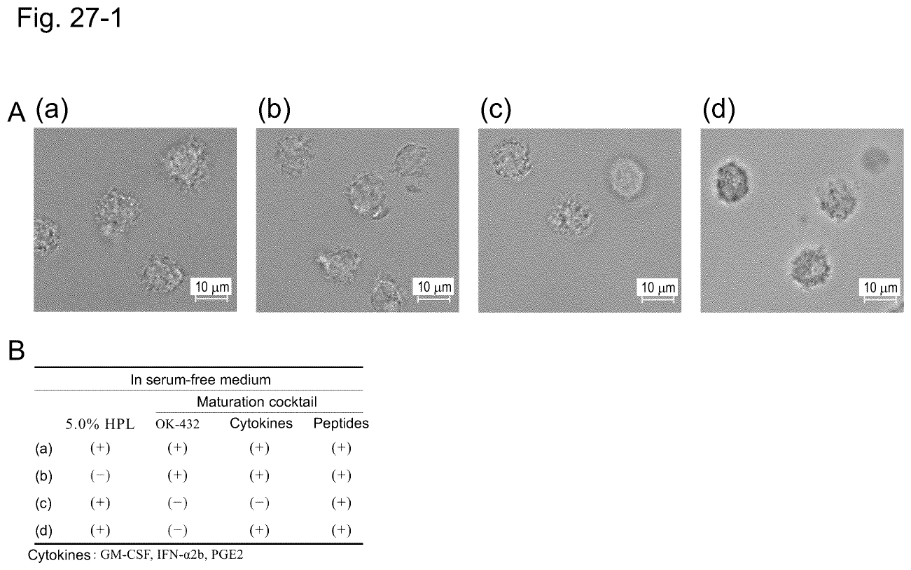

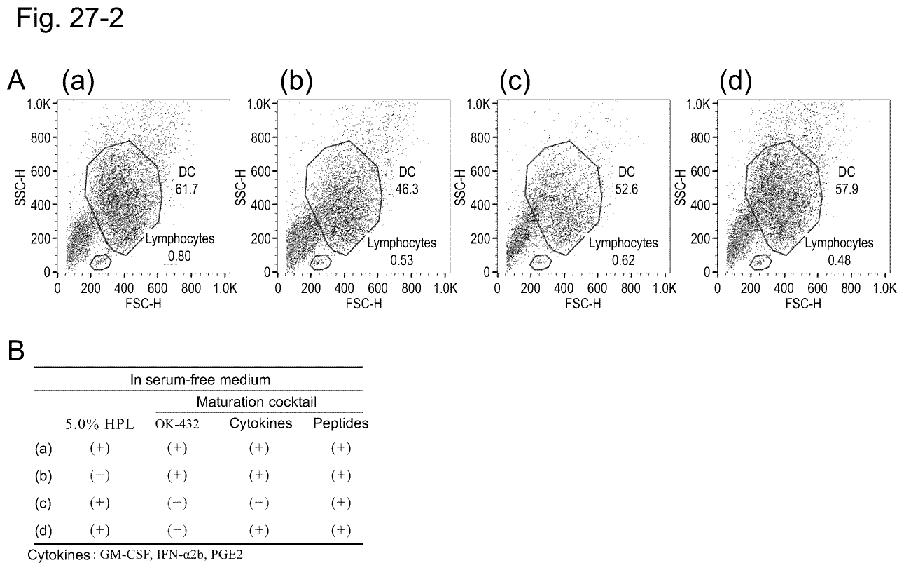

In Preliminary Test 5, necessity of reagents in maturation media in the process of HPL-IFN-DC preparation was evaluated (n=1). In the process of HPL-IFN-DC maturation, maturation cocktails of various compositions (maturation media) ((a) to (d)) were used. FIG. 27-1 shows (B) compositions of maturation cocktails used ((a) to (d)) and (A) microscopic images of IFN-DCs. As shown in FIG. 27-1, cytokines GM-CSF, IFN-α2b, and PGE2 were added to the maturation cocktails.

When maturation cocktails of various compositions ((a) to (d)) were used in the process of HPL-IFN-DC maturation, dendritic processes were observed, and no clear differences were observed in cell morphology.

The contamination rate of lymphocyte at the time of IFN-DC collection was evaluated by flow cytometry. The results are shown in FIG. 27-2. In Preliminary Test 5, HPL (5 (v/v) %) was used in the step of adhesion and separation of monocytes. Accordingly, the contamination rate of lymphocyte was lower than 1% (n=1).

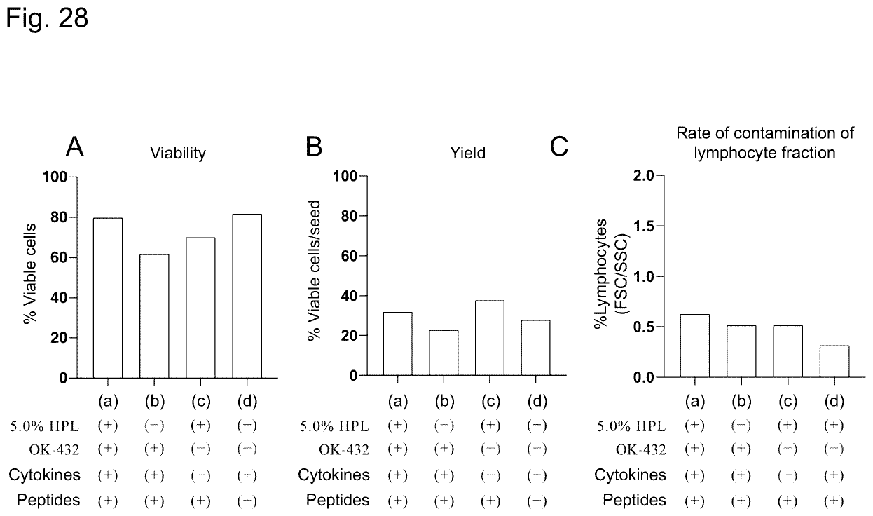

FIG. 28 shows (A) the cell viability of IFN-DCs prepared under various conditions, (B) the yield, and (C) the contamination rate of lymphocyte. In the process of maturation, HPL, OK432, or cytokines were excluded, and the low cell viability and the low yield were thus observed (n=1).

Phenotypes of HPL-IFN-DCs prepared with the use of maturation media were evaluated by flow cytometry (n=1).

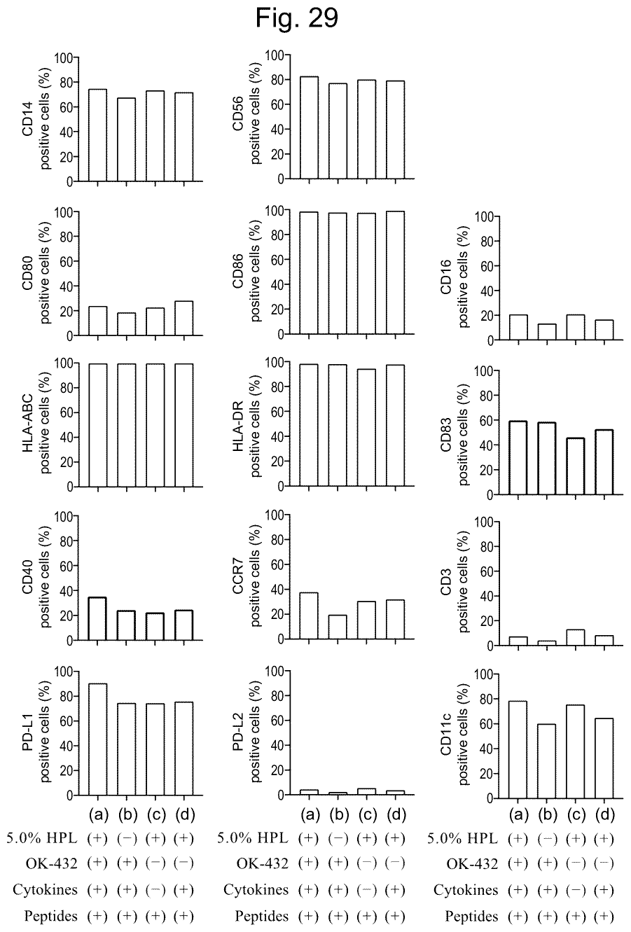

FIG. 29 shows the results of phenotype analysis of IFN-DCs prepared under various conditions. In comparison with (a), CD80, CCR7, CD40, and CD11c expression levels were found to be lower in the maturation media not supplemented with HPL (b). On the basis of comparison between (a) and (c), expression levels of CD83, CD40, and CCR7 serving as indicators of the antigen-presenting ability of DCs were lowered by removing cytokines and OK432 from the maturation medium.

On the basis of the results attained in Preliminary Test 5, the presence or absence of HPL at the time of maturation in the step of HPL-IFN-DC preparation was found to affect the cell viability and the yield. When OK432 and cytokines were removed from the maturation medium, in addition, lowered expression of CD83 and CD40 involved in the antigen presenting ability and lowered expression of CCR7 involved in the lymphocyte induction ability were observed. This indicates that HPL-IFN-DC functions would be deteriorated. In the process of HPL-IFN-DC preparation, accordingly, it is necessary to add HPL, cytokines, and OK432.

### Preliminary Test 6

On of the features of IFN-DCs is the cytotoxic activity to kill cancer cells. The cytotoxic activity of IFN-DCs prepared with the use of the HPL-supplemented DCO-K medium (HPL-IFN-DCs) was examined. In order to evaluate as to whether or not the storage conditions of the starting materials of HPL-IFN-DCs (PBMCs) would affect the cytotoxic activity, the cytotoxic activity of HPL-IFN-DCs prepared from fresh PBMCs was compared with that of HPL-IFN-DCs prepared from cryopreserved PBMCs.

### Preliminary Test 6:

Cells of the cancer cell line, the chronic myeloid leukemia cell line, K562 (ATCC, Manassas, VA. U.S.A.) were suspended at 1×10⁶ cells/ml in PBS containing FBS (0.1 (v/v) %) supplemented with a fluorescent dye (carboxyfluorescein succinimidyl ester (CFSE), 5 μM, Molecular Probes), subjected to a reaction at 37° C. for 10 minutes, and then washed in the AIM-V medium. With the use of the AIM-V medium containing FBS (10 (v/v) %). HPL-IFN-DCs (5×10⁵ cells, effector (E) cells, unstained) were mixed with CFSE-stained cancer cells (K562, target (T) cells) at a ratio of E:T of 50:1, and the resultant was then subjected to a reaction at 37° C. for 18 hours. The cells were washed 2 times with FACS flow buffer, stained with 2 μg/ml of propidium iodide (PI; Sigma-Aldrich Co. LLC., Tokyo, Japan) for 10 minutes to identify dead cells, and then analyzed by flow cytometry. The proportion of PI-positive cells in the CFSE-positive K562 cells excluding naturally dead cells was evaluated as the cytotoxic activity (% cytotoxicity) (n=2).

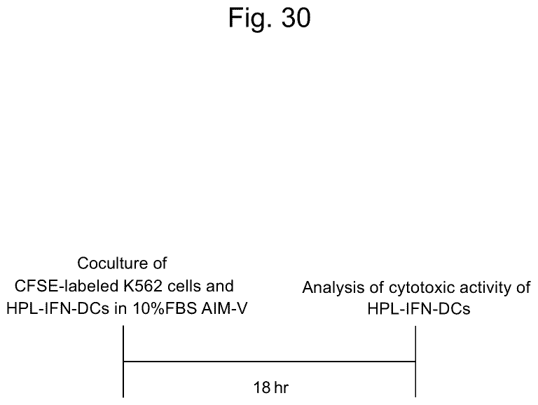

FIG. 30 shows a protocol of Preliminary Test 6.

In the past, Koya et al. reported that IFN-DCs prepared from PBMCs obtained from a patient by purifying monocytes with the aid of CD14 microbeads (Miltenyi Biotec, Bergisch Gladbach, Germany) with the use of a serum-free medium (AIM-V) had cytotoxic activity (Koya et al., Scientific Report 7, Article number: 42145: 2017).

Thus, the cytotoxic activity of IFN-DCs prepared with the use of an HPL-supplemented serum-free medium (DCO-K) was assayed. Since the cytotoxic activity of IFN-DCs may be lost because of cryopreservation, the influence of freezing on the cytotoxic activity was additionally evaluated (n=2).

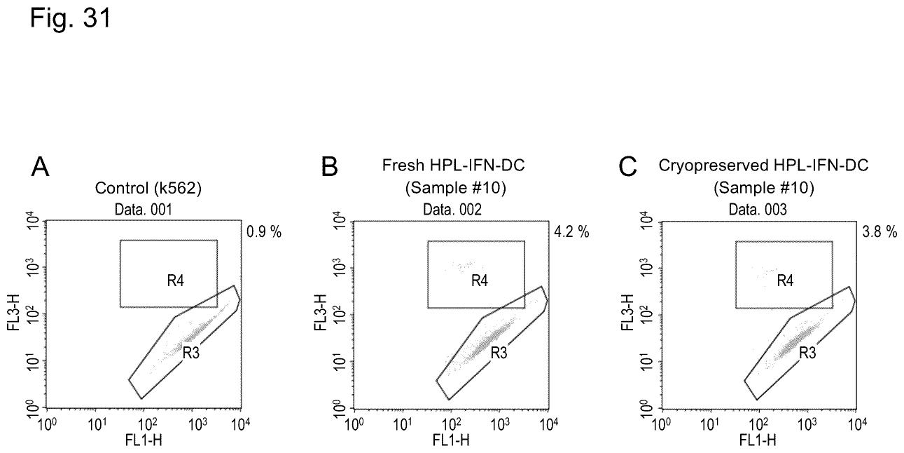

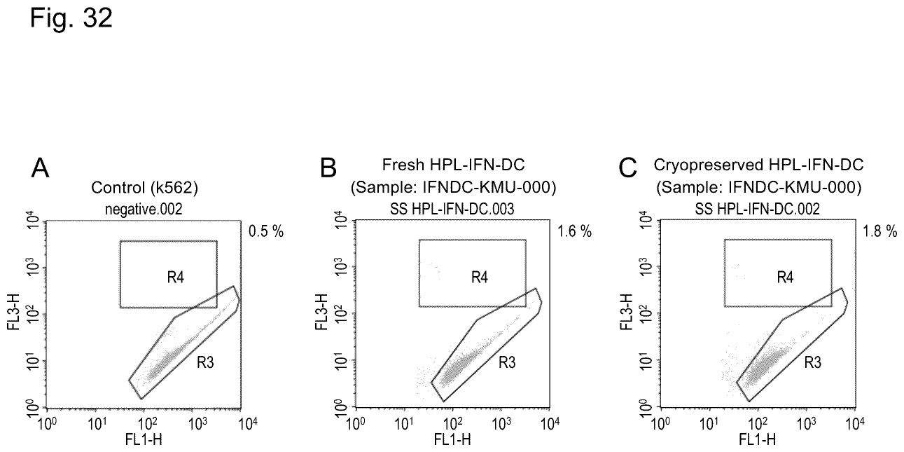

FIG. 31 and FIG. 32 show the results of cytotoxic activity assays on HPL-IFN-DCs prepared with fresh or cryopreserved PBMCs. FIG. 31 shows the results attained with the use of Sample #10 and FIG. 32 shows the results attained with the use of the sample IFNDC-KMU-000. A shows the results attained with the use of a control (K562), B shows the results attained with the use of fresh PBMCs. and C shows the results attained with the use of cryopreserved PBMCs. In FIG. 31, the proportion of fresh HPL-IFN-DCs was 4.2%, and that of frozen HPL-IFN-DCs was 3.8%. In FIG. 32, the proportion of fresh HPL-IFN-DCs was 1.6%, and that of frozen HPL-IFN-DCs was 1.8%.

HPL-IFN-DCs prepared with the use of the HPL-supplemented DCO-K medium exhibited the equivalent cytotoxic activity, regardless of cryopreservation, and no differences were observed.

In Preliminary Test 6, one of the features of IFN-DCs, that is, the cytotoxic activity, was found to be low in HPL-IFN-DCs. In the cases of HPL-IFN-DCs prepared from fresh or cryopreserved PBMCs, no differences were observed in terms of the cytotoxic activity. That is, the cytotoxic activity would not be affected by freezing of starting materials.

### Preliminary Test 7

In Preliminary Test 7, the CD8⁺ T cell-inducing ability of HPL-IFN-DCs was evaluated. IFN-DCs or HPL-IFN-DCs subjected to pre-pulsing with the cancer antigen, MART-1, prepared from a patient carrying HLA-A*02:01 (melanoma antigen recognized by T cell-1) (the AIM-V media were used as basal media) were mixed with the peripheral blood lymphocytes (1×10⁶ PBLs) at a ratio of 1:10, and culture was performed in the AIM-V media supplemented with IL-2 (5 ng/ml), IL-7 (5 ng/ml), and IL-15 (10 ng/ml) for 3 days. Subsequently, the AIM-V medium containing 10 (v/v) % ABS was added in accordance with the cell growth, IFN-DCs or HPL-IFN-DCs were added again on Day 7 and Day 14 from the initiation of culture, cells were collected on Day 21, and the antigen presenting ability was evaluated based on induction of MART-1-specific CD8⁺ T cells. The collected cells were stained with CD8-FITC, CD3-APC, and T-select HLA-A*0201 MART-1 tetramer-ELAGIGILTV-PE, and MART-1-specific CD8⁺ T cells were detected using a flow cytometer (n=1).

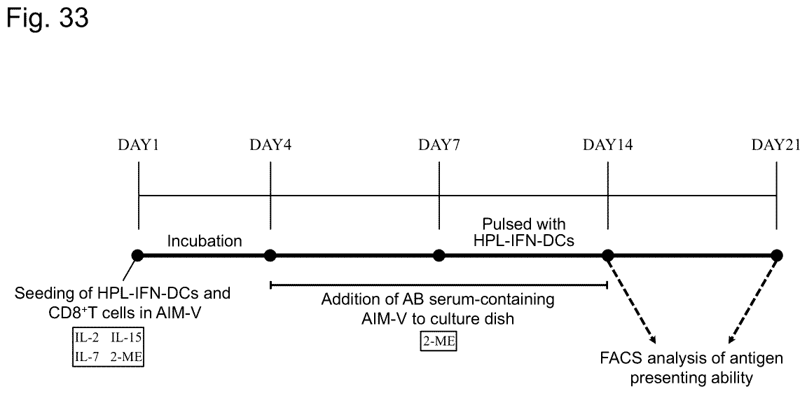

FIG. 33 shows a protocol of Preliminary Test 7.

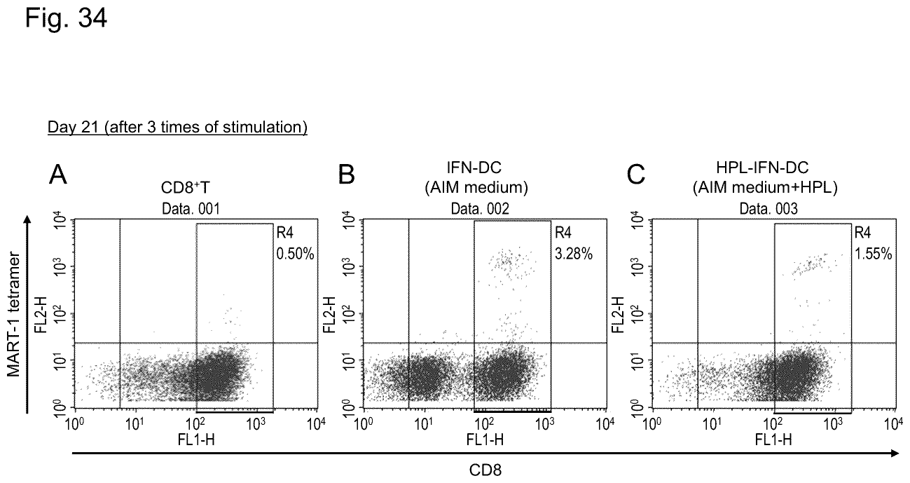

The cytotoxic T cell-inducing ability of HPL-IFN-DCs prepared with the use of the serum-free media (AIM-V) was analyzed by flow cytometry (n=1). The results are shown in FIG. 34. A shows the results of analysis of CD8+ T cells, B shows those of IFN-DCs. and C shows those of HPL-IFN-DCs. In comparison with IFN-DCs prepared with the use of the serum-free media (AIM-V), HPL-IFN-DCs prepared with the addition of HPL exhibited lower antigen presenting ability (IFN-DC: 3.28%; HPL-IFN-DC: 1.55%). The symbol “%” in dot plots indicates the proportion of MART-1-specific CTLs induction in CD8⁺ T cells.

IFN-DCs prepared with the addition of 5% (v/v) HPL to AIM-V exhibited the low MART-1-specific CTLs induction ability. This indicates that differences in compositions between the AIM-V medium and the DCO-K medium affect the antigen presenting ability of IFN-DCs.

### Conclusion of Preliminary Tests

Adequacy of a novel method for preparing IFN-DCs using monocytes was evaluated and a procedure was determined.

The method for IFN-DC preparation with the addition of HPL to a serum-free medium (DCO-K) was deduced to be an inventive and novel procedure based on the adhesion properties of monocytes (purification of starting materials) and the results attained in the procedure of differentiation induction and maturation of IFN-DCs in terms of the cell viability, the yield, and the purity (the contamination rate of lymphocyte).

The phonotypes of mature HPL-IFN-DCs as products constitute a homogeneous cell population of CD86⁺HLA-ABC⁺HLA-DR⁺. The proportion of positive cells for CD14 and CD56 cells was increased with the addition of HPL, and concentration-dependent expression of CD56⁺, CD80⁺, CD83⁺ cells was observed.

The DCO-K media supplemented with HPL at 1 to 10 (v/v) % were found to be applicable to preparation of IFN-DCs derived from monocytes.

In HPL-IFN-DCs, an increase was not observed in killer activity, regardless of CD56 expression.

When IFN-DCs were prepared with the use of the previously evaluated AIM-V medium supplemented with HPL, the antigen presenting ability of the IFN-DCs was lower than that attained with the use of the AIM-V medium without additives.

As a method for IFN-DC preparation intended for clinical application, as described above, a procedure comprising adhesion, differentiation induction, and maturation of monocytes with the use of the serum-free medium (DCO-K) in combination with 5 (v/v) % HPL was found to be optimal.

In Example 2 below (Main Test), examination was performed with reference to the procedure of preparation described above.

### [Example 2] Establishment of a Method for Monocyte Separation and a Method for IFN-DC Preparation Using a Serum-Free Media Supplemented with HPL (5 (v/v) %) (DCO-K)

This example was performed as main testing.

A protocol comprising preparing IFN-DCs with the use of a serum-free medium supplemented with HPL (5 (v/v) %) (DCO-K) in the step of separating monocytes from PBMCs obtained from a patient (30 minutes) and the step of differentiation and maturation was established based on the preliminary testing performed in Example 1.

### Established Protocol:

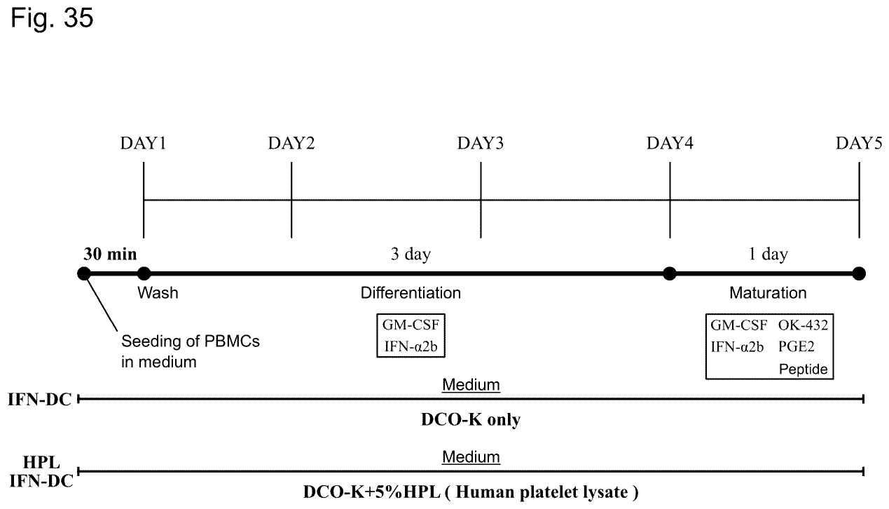

The peripheral blood mononuclear cells (PBMCs) collected from a patient by apheresis were seeded in an adhesion culture dish using a serum-free medium (DCO-K) supplemented with HPL (final concentration: 5 (v/v) %). Culture was performed at 37° C. in the presence of 5% CO₂ for 30 minutes to allow the cells to adhere to the bottom of the culture dish, and monocytes were separated from lymphocytes. Subsequently, adherent cells were induced to differentiate into IFN-DCs with the use of the DCO-K medium supplemented with 1 μg/ml PEG-Intron, 100 ng/ml GM-CSF, and HPL. The cells were collected 3 days after the initiation of differentiation and cultured using a maturation medium supplemented with various reagents (10 μg/ml OK432 and 10 ng/ml PGE2) and 20 μg/ml of tumor antigen peptide (WT1: Wilms tumor 1) in a low-adhesion culture dish for 18 to 24 hours for IFN-DC maturation. A protocol is shown in FIG. 35.

With the use of the HPL-IFN-DCs prepared by the established protocol, main testing was performed (n=6).

### Main Test 1

In Main Test 1, the cell viability, the rate of collection, and the purity were compared and examined between HPL-IFN-DCs and IFN-DCs (n=6).

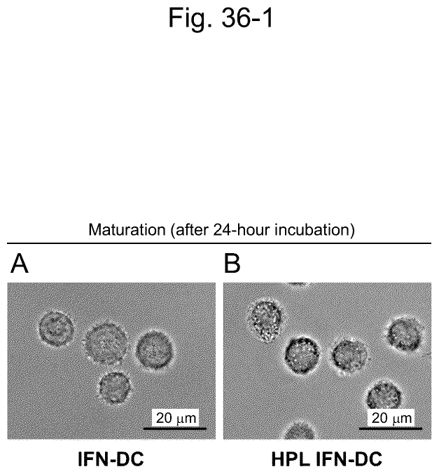

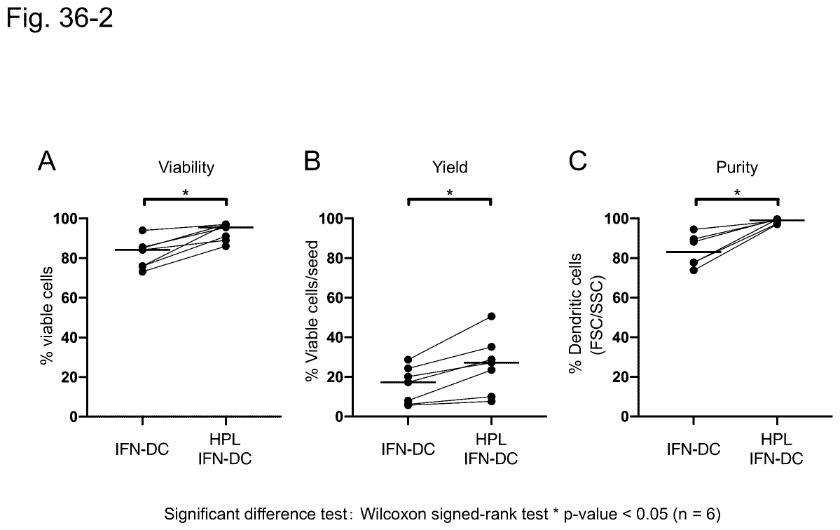

The peripheral blood mononuclear cells collected from a patient by apheresis were suspended in the DCO-K medium supplemented with HPL (5 (v/v) %) and the cell suspension was seeded in an adhesion culture dish. Culture was performed at 37° C. in the presence of 5% CO₂ for 30 minutes, and non-adherent cells were washed to separate the monocytes. Adherent cells were induced to differentiate into IFN-DCs with the addition of a differentiation induction medium supplemented with PEG-Intron and GM-CSF. The cells were collected 3 days after the initiation of differentiation, suspended in a maturation medium supplemented with various reagents (PEG-Intron, GM-CSF, PGE2, and OK432), and, at the same time, seeded in a low-adhesion culture dish for maturation. The cells were collected 24 hours later, and cell morphology was observed under a phase contrast microscope. FIG. 36-1 shows cell observation images. A shows observation images of IFN-DCs and B shows observation images of HPL-IFN-DCs. The dendritic processes observed indicate differentiation into DCs. The presence or absence of HPL did not affect the cell morphology.

In addition, the cell viability, the yield, and the purity of IFN-DCs collected after maturation were compared. The results are shown in FIG. 36-2. A shows the cell viability, B shows the yield, and C shows the purity. Significant increases were observed in the IFN-DCs prepared with the addition of HPL (HPL-IFN-DCs) (viability: IFN-DCs: 84.2%; HPL-IFN-DCs: 95.5%; yield: IFN-DCs: 14.1%; HPL-IFN-DCs: 25.4%; purity: IFN-DCs: 83.1%; HPL-IFN-DCs: 99.1%). The results of Main Test 1 demonstrate that the cell viability, the yield, and the purity of the IFN-DCs prepared with the addition of HPL (5 (v/v) %) would be high.

### Main Test 2

In Main Test 2, the influence of HPL on the IFN-DC phenotype was analyzed by flow cytometry (n=6).

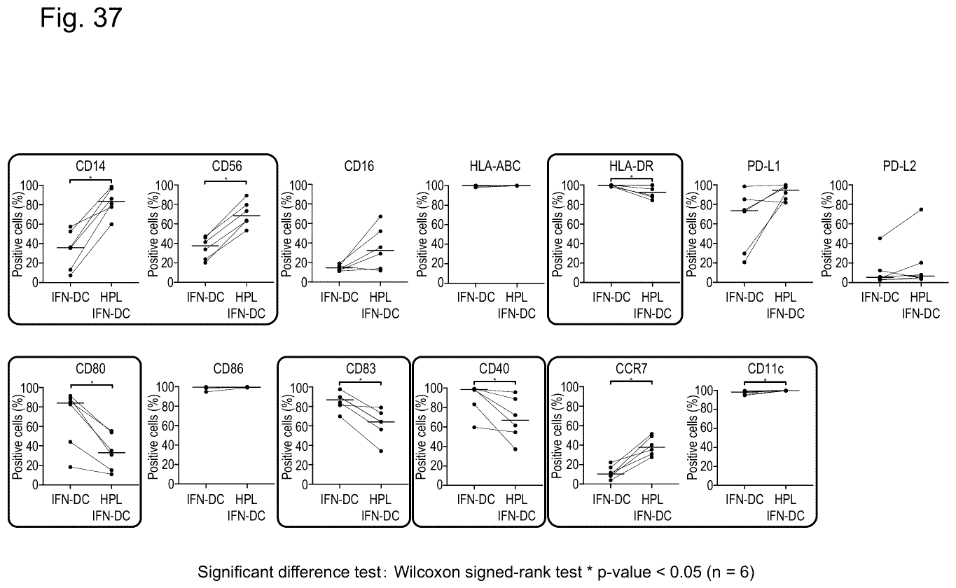

The results are shown in FIG. 37. In comparison with IFN-DCs, expression levels of a monocyte marker CD14, a cell adhesion molecule CD56, CCR7 (CD197) that promotes migration to the lymph node, and a dendritic cell marker CD11c were found to be elevated to a significant extent in the HPL-IFN-DCs prepared with the addition of HPL. In addition, expression levels of costimulatory molecules CD80 and CD40 involved in the antigen presenting ability to T cells, a dendritic cell maturation marker CD83, and HLA-DR involved in antigen presentation were found to be lowered to a significant extent.

### Main Test 3

In Main Test 3, the antigen phagocytic ability and the degradation ability of HPL-IFN-DCs and IFN-DCs were evaluated by flow cytometry using FITC-dextran and DQ-ovalbumin (DQ-OVA).

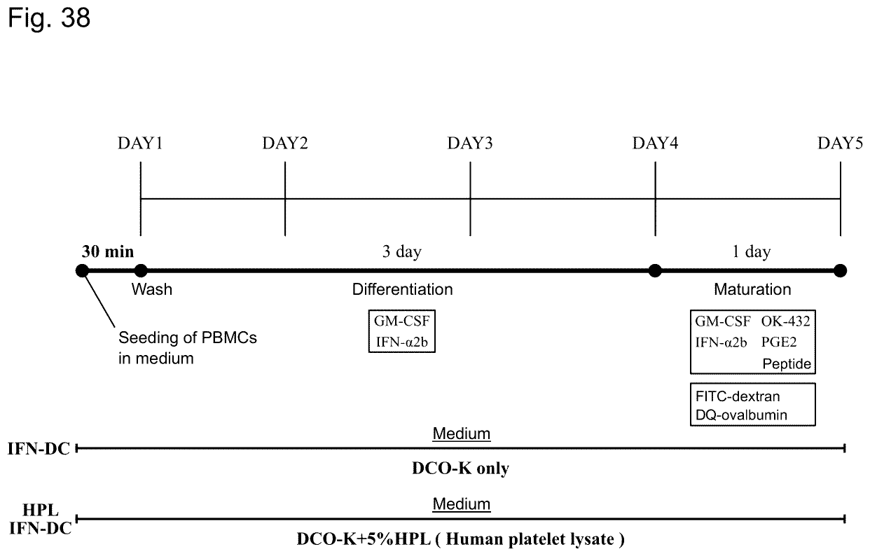

In the maturation step, 100 μg/ml FITC-Dextran (Molecular Probes, Eugene, OR, U.S.A) and 10 μg/ml DQ ovalbumin (Molecular Probes) were added to a maturation medium, and culture was performed for 24 hours. Thereafter, the IFN-DCs or HPL-IFN-DCs were washed 2 timed with PBS, resuspended in 1 (v/v) % FBS-PBS, and subjected to evaluation in terms of the phagocytic ability and the degradation ability by flow cytometry (n=6). A protocol is shown in FIG. 38.

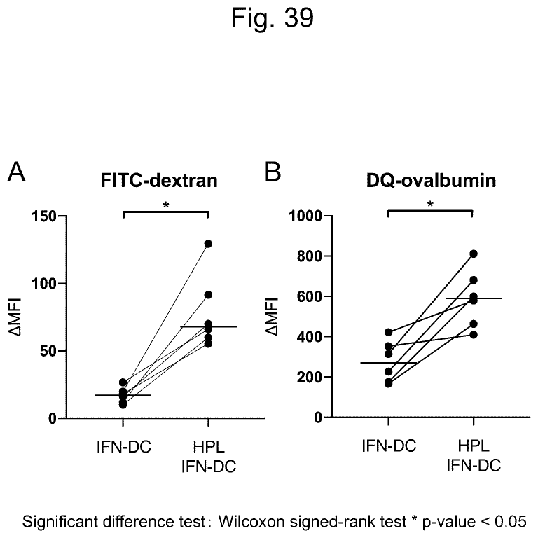

The antigen phagocytic ability and the antigen degradation ability of IFN-DCs and HPL-IFN-DCs were examined by flow cytometry using FITC-dextran and DQ-ovalbumin (n=6). The results are shown in FIG. 39. FITC-dextran incorporation and the DQ-OVA degradation ability were inspected, and the antigen phagocytic ability and the antigen degradation ability were represented by dot plots of ΔMFI. A shows the results attained using FITC-dextran and B shows the results attained using DQ-ovalbumin.

In comparison with IFN-DCs, the antigen phagocytic ability and the degradation ability of the IFN-DCs supplemented with HPL were higher (FITC-dextran ΔMFI: IFN-DCs: 17.1; HPL-IFN-DCs: 68.0; DQ-Ovalbumin ΔMFI: IFN-DCs: 270.9; HPL-IFN-DCs: 589.7).

### Main Test 4

In Main Test 4, the ability of IFN-DCs and HPL-IFN-DCs for producing various cytokines was evaluated.

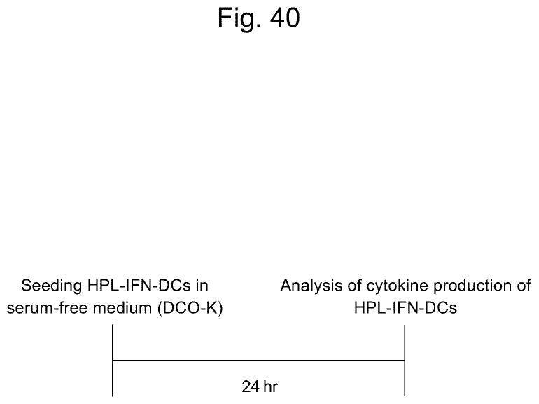

IFN-DCs and mature HPL-IFN-DCs prepared in accordance with the established protocol were suspended in the DCO-K medium to a cell concentration of 1×10⁶ cells/ml, and the cell suspension was seeded in a culture dish. Culture was performed at 37° C. in the presence of 5% CO₂ for 24 hours, and the culture supernatant was then collected. The collected culture supernatant was subjected to assays of various cytokines (IL-6, IL-10, IL-12 (p70), IFN-γ, and TNF-α) using the Bio-plex assay kit (Bio-Rad Labs). TGF-0 was assayed using the human TGF-beta 1 Quantikine ELISA Kit (R & D systems) (n=6). A protocol is shown in FIG. 40.

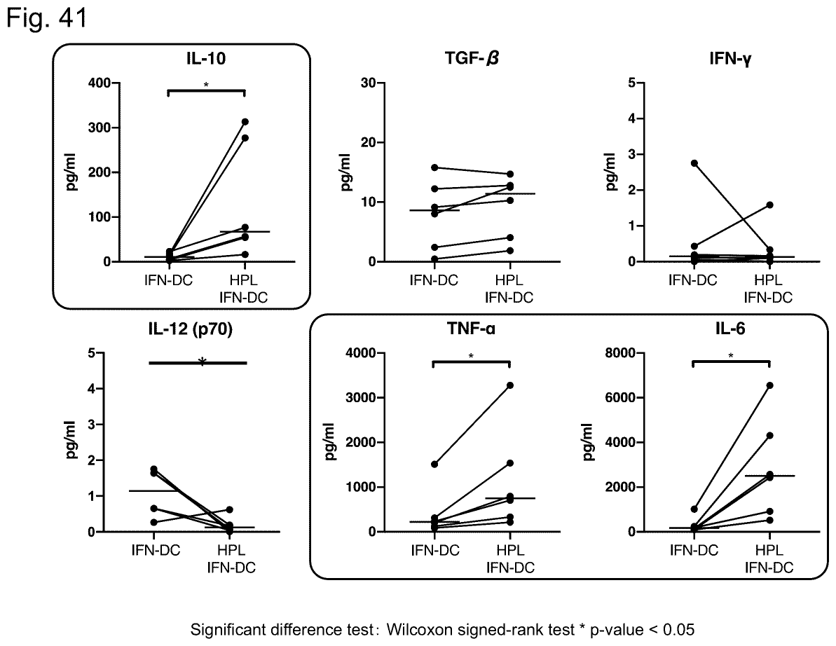

Subsequently, cytokines (IL-10, TGF-β, IFN-γ, TNF-α, IL-12 (p70), and IL-6) involved in induction of cytotoxic T cells secreted from HPL-IFN-DCs were assayed using the Bio-plex assay kit (Bio-Rad Labs) (n=6). The results are shown in FIG. 41.

In comparison with IFN-DCs, the amount of production of the Th1 cytokine IL-12 (p70) that would promote cytotoxic T cell induction was significantly lower in HPL-IFN-DCs (IL-12 production: IFN-DCs: 1.1 μg/ml; HPL-IFN-DCs: 0.18 μg/ml), and no changes were observed in IFN-γ exhibiting similar activity (IFN-γ production: IFN-DCs: 0.59 μg/ml; HPL-IFN-DCs: 0.38 μg/ml). In contrast, the amount of production of the Th2 cytokines IL-10 and TGF-β that would suppress cytotoxic T cell induction was increased in HPL-IFN-DCs (IL-10 production: IFN-DCs: 11.47 μg/ml; HPL-IFN-DCs: 132.7 μg/ml; TGF-β production: IFN-DCs: 8.02 μg/ml; HPL-IFN-DCs: 9.38 μg/ml). The amount of secretion of TNF-α and IL-6 involved in induction of inflammation reactions and activation and differentiation of T cells was significantly increased in HPL-IFN-DCs (IL-6 production: IFN-DCs: 302.3 μg/ml; HPL-IFN-DCs: 2883 μg/ml; TNF-α; IFN-DCs: 412.5 μg/ml; HPL-IFN-DCs: 1144.4 μg/ml).

HPL was found to vary the amounts of Th1/Th2 cytokines produced from IFN-DCs.

### Main Test 5

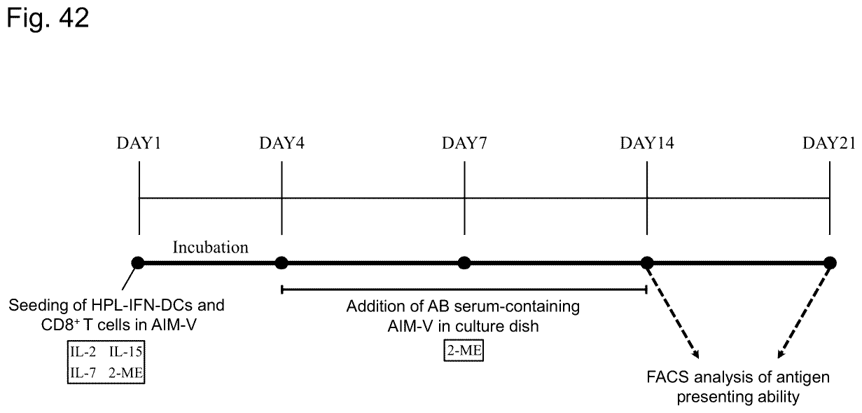

In Main Test 5, the MART-1-specific CD8⁺ T cell-inducing ability of IFN-DCs and that of HPL-IFN-DCs were evaluated. A protocol is shown in FIG. 42.

The cytotoxic T cell-inducing ability of HPL-IFN-DCs prepared with the use of the HPL-supplemented DCO-K medium was evaluated (n=6).

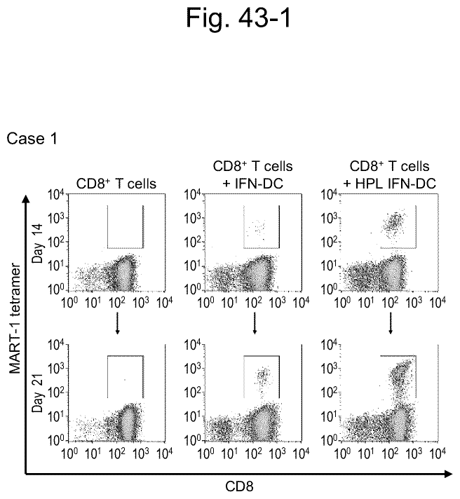

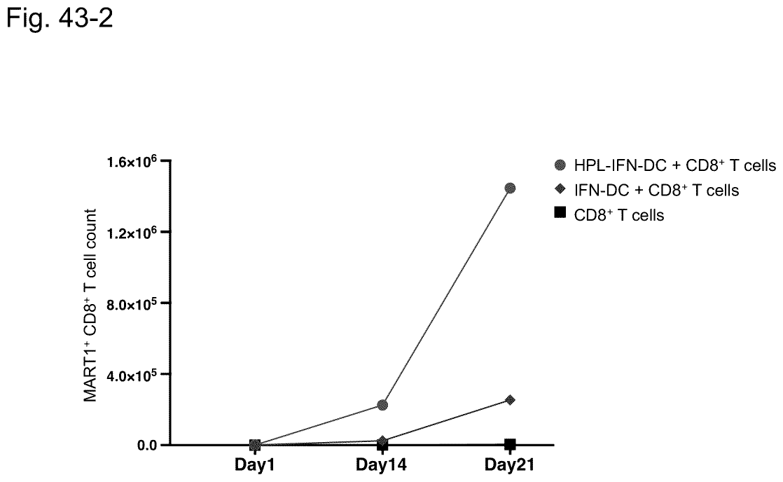

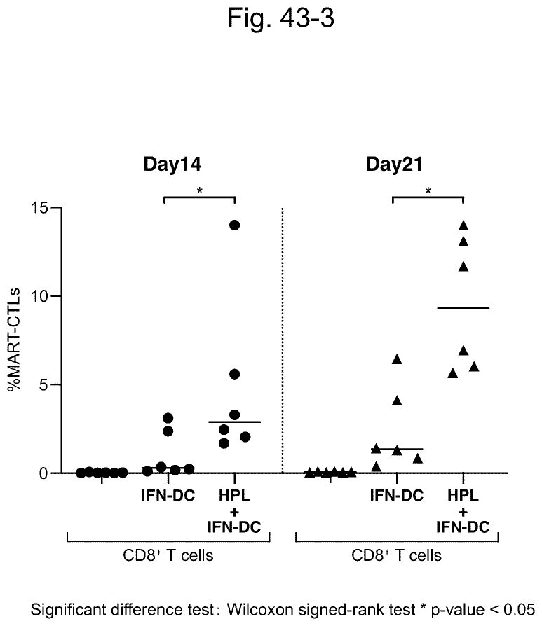

IFN-DCs and HPL-IFN-DCs pre-pulsed with MART-1 (melanoma antigen recognized by T cell-1) peptides were subjected to coculture with CD8-positive T cells, and MART-1-specific cytotoxic T cells were detected by flow cytometry on Day 14 and Day 21. FIG. 43-1 shows the results of analysis by flow cytometry, FIG. 43-2 shows the number of MART-1-specific CD8⁺ T cells in each treatment group, and FIG. 43-3 shows the proportion of MART-1-specific CD8⁺ T cells (MART-1-CTL, MART-1-specific CTL-positive cells). In comparison with IFN-DCs, MART-1-specific cytotoxic T cell induction was increased to a significant extent in HPL-IFN-DCs on Day 14 and Day 21 (the median of the positive cell number of MART-1 tetramer+CTLs on Day 14: CD8⁺ T cells, 1.37×10³ cells; CD8⁺ T cells+IFN-DCs, 2.45×10⁴ cells; CD8⁺ T cells+HPL IFN-DCs, 2.25×10⁵ cells; the median of the positive cell number of MART-1 tetramer+CTLs on Day 21: CD8⁺ T cells, 3.64-10³ cells; CD8⁺ T cells+IFN-DCs, 2.54×10⁵ cells; CD8⁺ T cells+HPL IFN-DCs, 1.45×10⁶ cells; n=6).

The cytotoxic T cell-inducing ability of IFN-DCs and that of HPL-IFN-DCs were examined by the intra-group significant difference test and compared (comparison was performed selectively on Day 14 and Day 21).

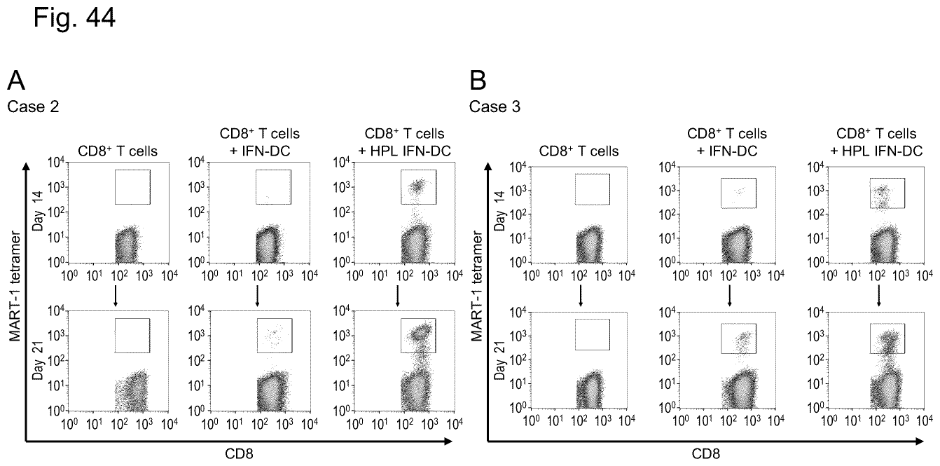

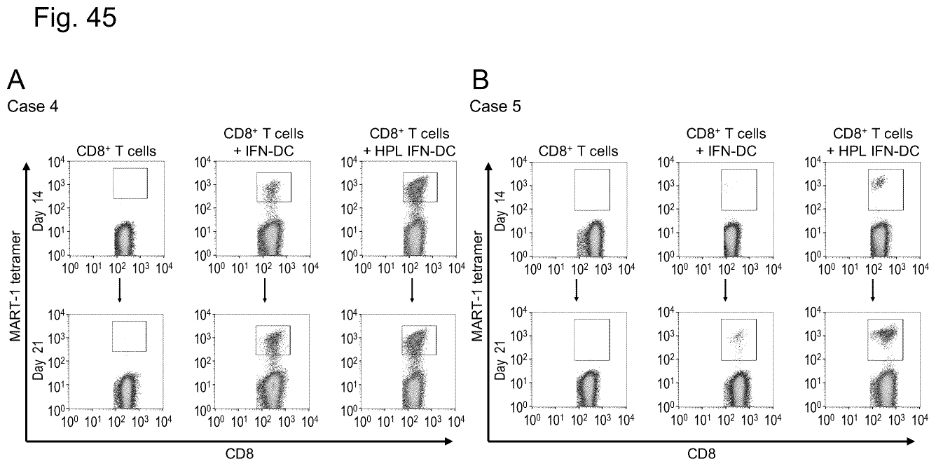

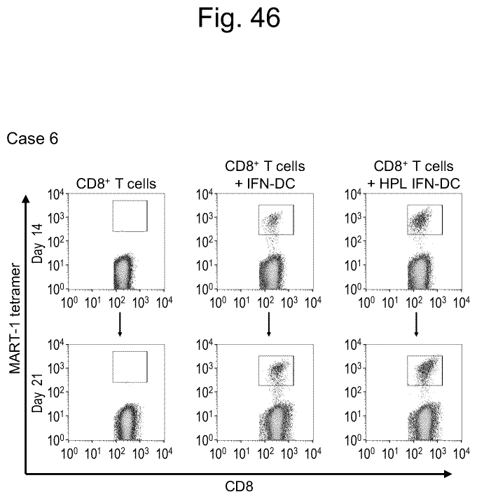

Five cases (Case 2, Case 3, Case 4, Case 5, and Case 6) are shown in the forms of dot plot charts in FIG. 44 (A: Case 2, B: Case 3), FIG. 45 (A: Case 4, B: Case 5), and FIG. 46 (Case 6) (formats of charts are as described above).

### Main Test 6

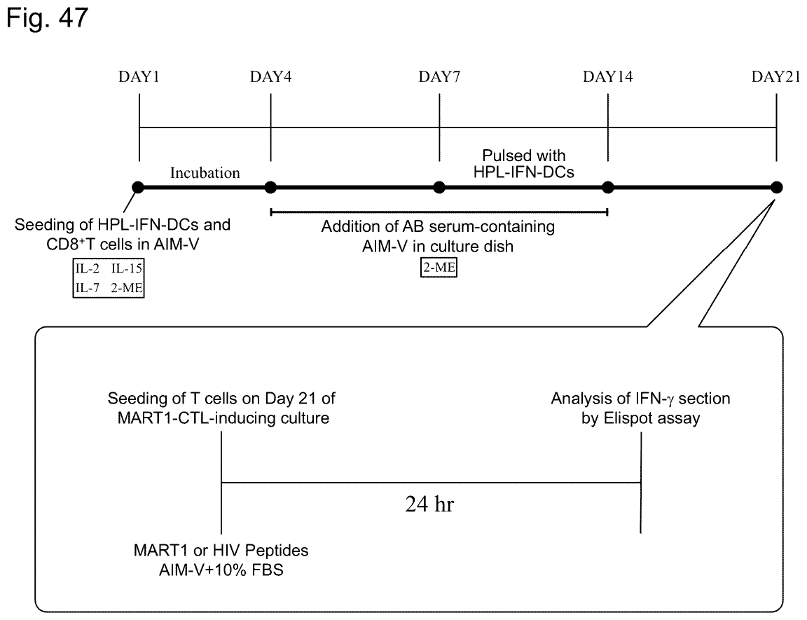

The antigen-specific IFN-γ-producing ability of cytotoxic T cells induced by IFN-DCs and that of HPL-IFN-DCs were evaluated by the Elispot assay (n=6). A protocol is shown in FIG. 47.

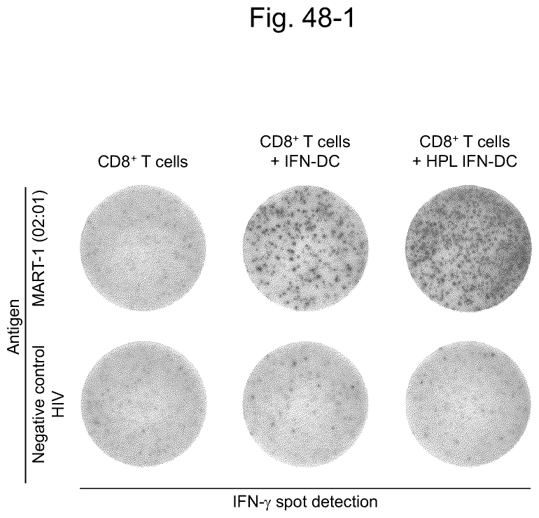

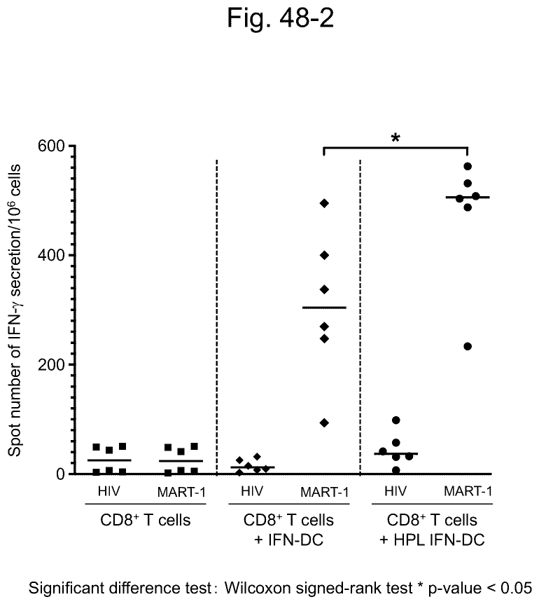

FIG. 48-1 shows spot images, and FIG. 48-2 shows the amount of IFN-γ secretion (the amount of production). In comparison with IFN-DCs, the amount of antigen-specific IFN-γ secretion from cytotoxic T cells was increased to a significant extent in HPL-IFN-DCs.

### Summary of Results of Main Testing

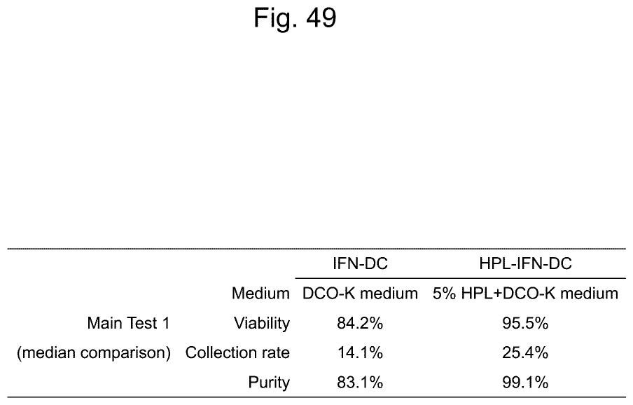

FIGS. 49 to 51 each show precise numerical values obtained in Main Tests 1 to 6.

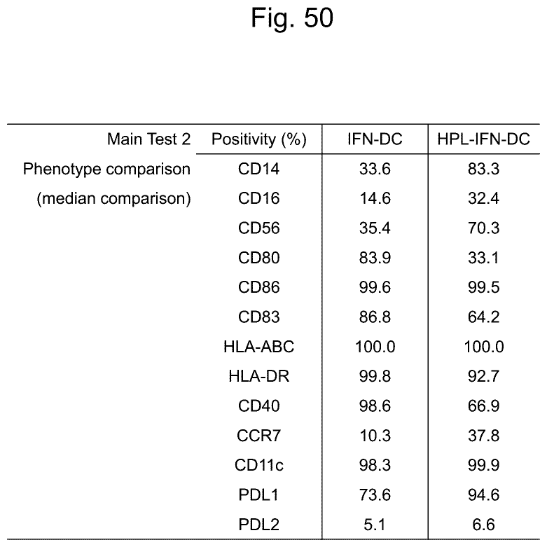

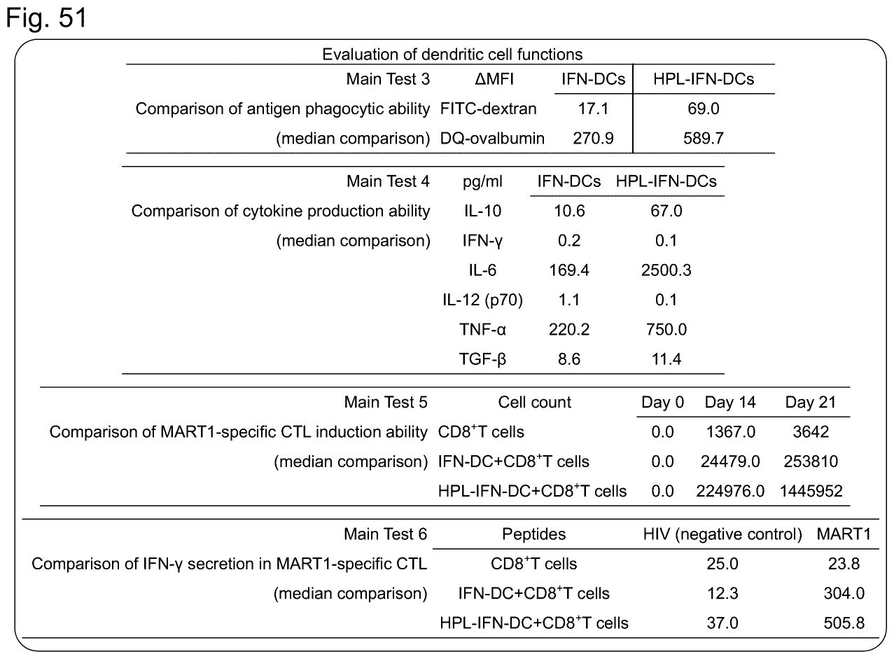

As shown in FIG. 49, HPL-IFN-DCs were found to be excellent in terms of the cell viability, the rate of collection, and the purity. As shown in FIG. 50, HPL-FN-DCs have traits that have not been heretofore found in DCs. FIG. 51 shows the results of functional evaluation of DCs. As a result of functional evaluation of HPL-IFN-DCs, the antigen phagocytic ability, the antigen degradation ability, the cytokine-producing ability, and the cytotoxic T cell-inducing ability thereof were found to be higher than those of IFN-DCs.

On the basis of the results of the main tests, IFN-DCs prepared with the use of a serum-free medium supplemented with 5 (v % v) % HPL (DCO-K) were found to show improvement in monocyte separation in the production process and improvement in the cell viability, the yield, and the purity of the final product. On the basis of the antigen presenting ability, the phagocytic ability, and the degradation ability determined by functional evaluation of dendritic cells, in addition, the method of preparing IFN-DCs according to the present invention can be concluded as an inventive and novel method.

On the basis of phenotype analysis of HPL-IFN-DCs, HPL-IFN-DCs were found to constitute a homogeneous cell population of CD14⁺, CD56⁺, CD86⁺, CCR7⁺, and HLA-ABC/DR⁺, and the proportion of CD56⁺, CD80⁺, and CD83⁺ cells were elevated in an HPL-concentration-dependent manner. That is, HPL-IFN-DCs were found to have novel traits outside the scope of DC fractions that has heretofore been reported.

In HPL-IFN-DCs, the proportion of the costimulatory factors CD80 and CD40 involved in the antigen presenting ability to T cells and that of the dendritic cell maturation marker CD83 were lowered, the amount of secretion of a Th1 cytokine inducing cytotoxic T cells, IL-12 (p70), was lowered, and the amount of secretion of an inhibitory Th2 cytokine, IL-10, was increased. However, HPL-IFN-DCs were found to have the high antigen presenting ability and significant cytotoxic T cell-inducing ability.

According to a method for preparing IFN-DCs with the use of the HPL-supplemented serum-free medium (DCO-K), improvement was observed in the cell viability, the rate of collection, and the purity, and the antigen presenting ability, the antigen degradation ability, and the antigen phagocytic ability of such IFN-DCs were superior to those of the IFN-DCs prepared without the addition of HPL. Thus, use thereof for novel DC vaccines that are effective for cancer immunotherapy and infection control can be expected.

### [Example 3] WT1 Peptide-Pulsed IFN Dendritic Cell Vaccines

### Production of IFN-DCs Using HPL

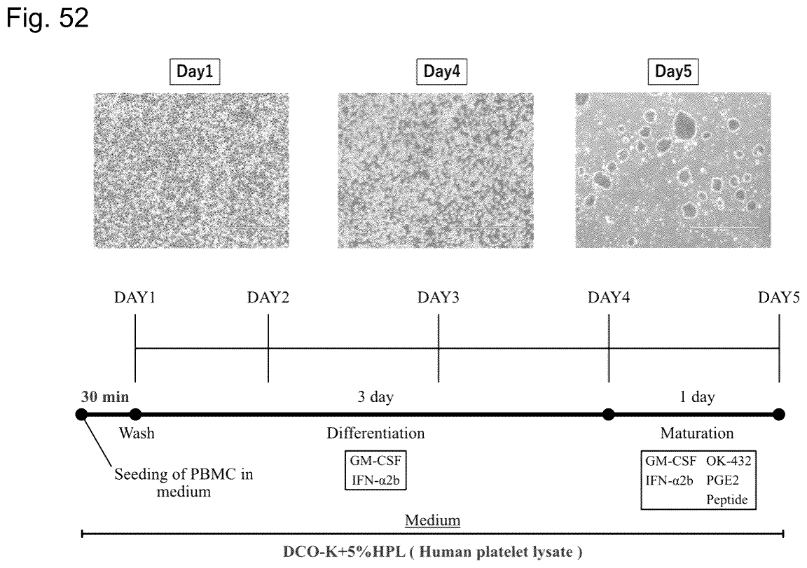

The peripheral blood mononuclear cells (PBMCs) were suspended in a medium, the cell suspension was seeded in a dish, non-adherent cells were removed by washing 30 minutes later, and the adherent monocytes were induced to differentiate using GM-CSF and IFN-α. OK-432, PGE2, and peptides were added on Day 4, and cells were collected 18 to 24 hours thereafter. A protocol is shown in FIG. 52. On Day 5, significant cluster formation, which is a feature of dendritic cells, was observed.

Selective Adhesion Culture of Monocytes when Preparing IFN Dendritic Cells Using HPL

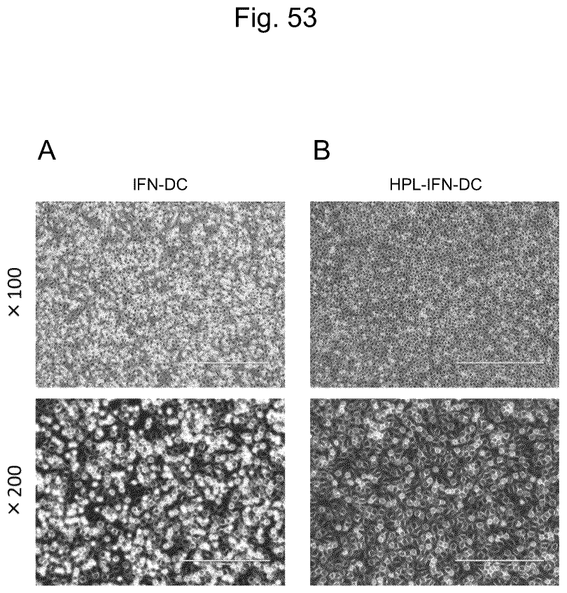

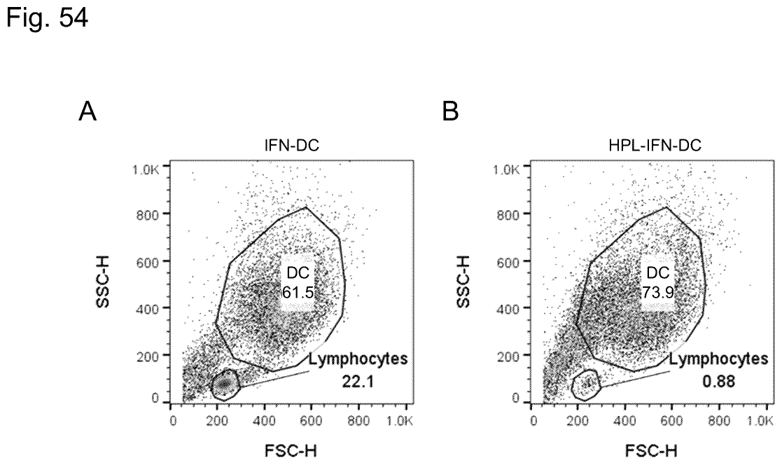

The AIM-V medium that had been used in conventional techniques was a reagent for research purposes, and such medium was not manufactured or managed in accordance with the clinical standard. Accordingly, culture was performed using a GMP-grade DCO-K medium (a serum-free medium with a known composition). With the use of HPL, monocytes can more selectively adhere to the culture vessel, compared with the case in which only the DCO-K medium is used at the time of seeding of the peripheral blood mononuclear cells. FIG. 53 shows culture conditions of dendritic cells. FIG. 53A shows IFN-DCs prepared without the use of HPL and FIG. 53B shows IFN-DCs with the use of HPL (HPL-IFN-DCs). FIG. 54 shows the results of flow cytometry. On the basis of flow cytometry images, contamination of lymphocyte fractions was found to be suppressed in the IFN-DCs (HPL-IFN-DCs) prepared with the use of HPL (FIG. 54A) to a significant extent, compared with the IFN-DCs prepared without the use of HPL (FIG. 54B) (IFN-DCs: 22.1%; HPL-IFN-DCs: 0.88%).

### Phenotype Analysis of HPL-IFN-DCs

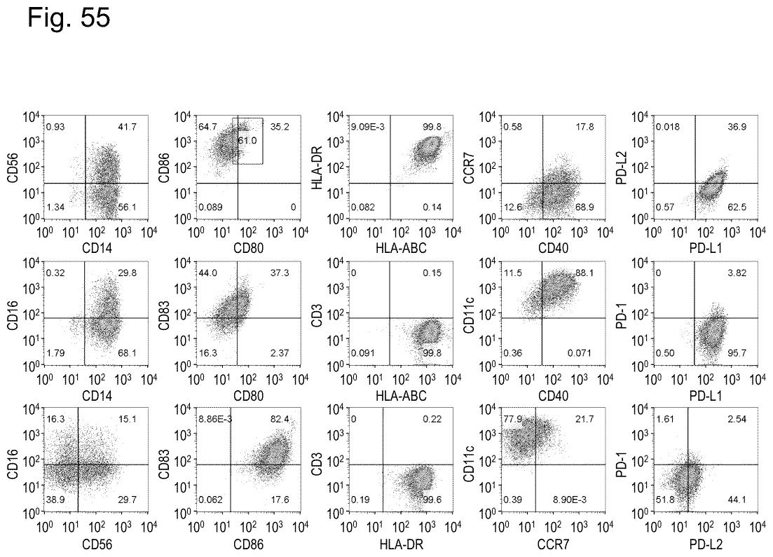

Monocytes were allowed to selectively adhere with the use of HPL, induced to differentiate with the use of GM-CSF and IFN-α, allowed to mature with OK432 or PGE2, and then subjected to phenotype observation using a flow cytometer. The results are shown in FIG. 55. Expression of cell surface markers reported concerning IFN-dendritic cells; i.e., CD11c, CD40, CD56, CD80, CD83, CD86, HLA-ABC, and HLA-DR, was observed.

### Induction of MART-1 Antigen-Specific Cytotoxic T Cells by IFN-DCs or HPL-IFN-DCs

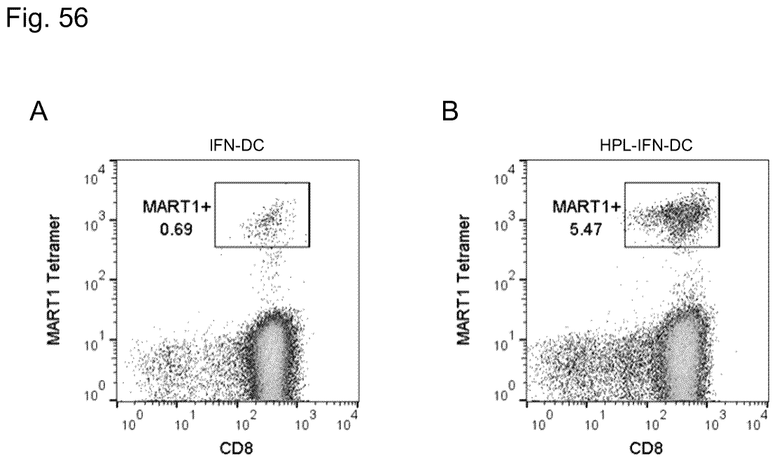

IFN-DCs or HPL-IFN-DCs comprising MART-1 26-35 A27L peptides incorporated therein were co-cultured with CD8+ T cells to perform the in vitro CTL induction test. The MART-1-specific cytotoxic T lymphocytes (CTLs) were detected 21 days after the initiation of the culture. The results are shown in FIG. 56. In comparison with IFN-DCs (FIG. 56A), induction of MART-1-specific CTLs was observed at a higher level in HPL-IFN-DCs (FIG. 56B) (IFN-DCs: 0.69%; HPL-IFN-DCs: 5.47%).

### Comparison of WT1-CTL Induction by WT1-Supplemented IL-4-DCs or HPL-IFN-DCs

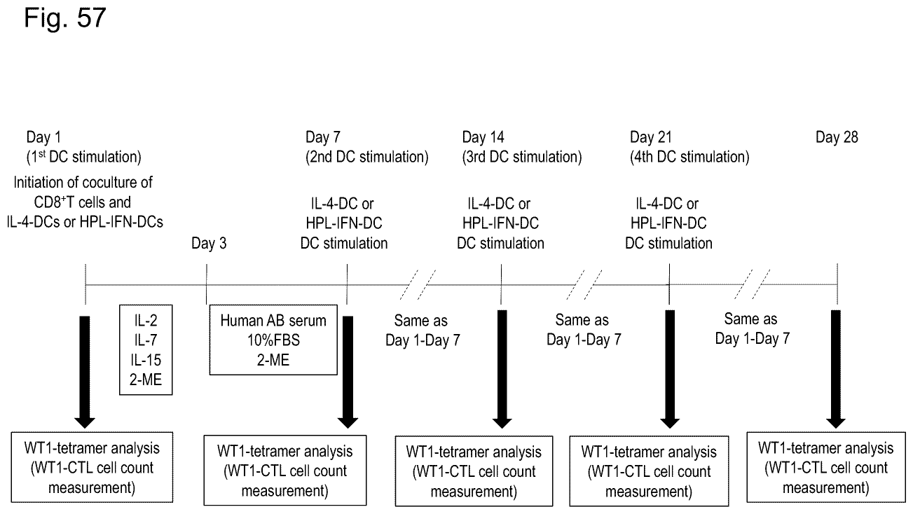

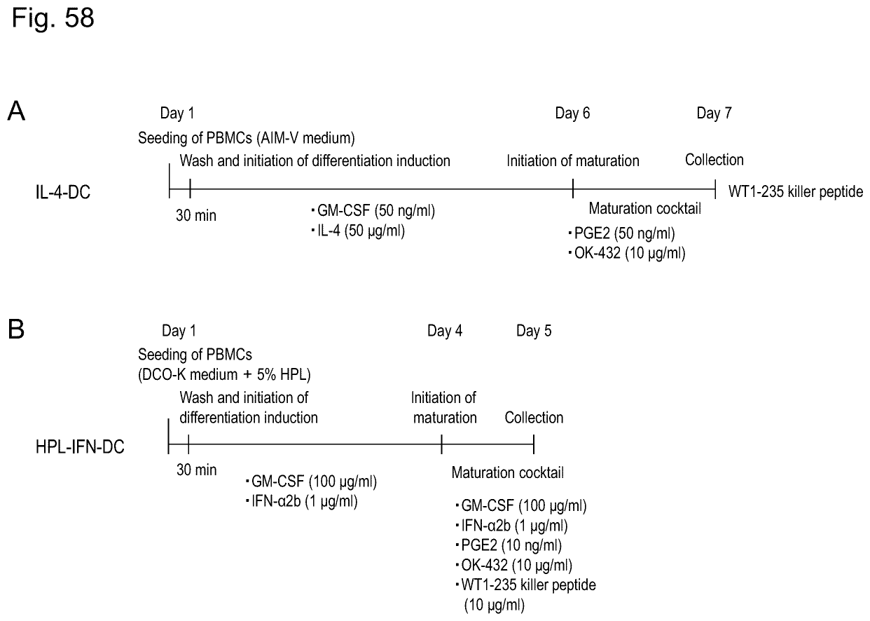

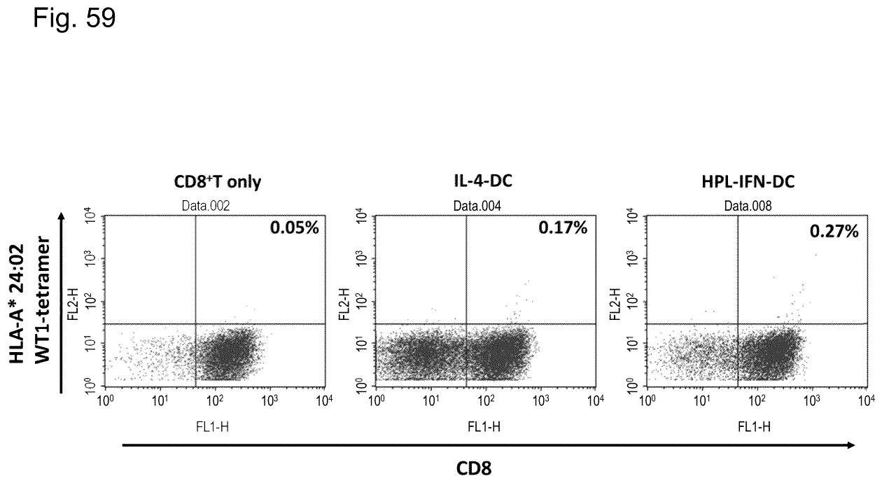

DCs comprising the WT1 antigens incorporated therein and CD8⁺ T cells were subjected to the in vitro CTL induction test, cells were collected 21 days after the initiation of the culture, and the proportion of WT1-CTL induction determined by the WT1-tetramer analysis was evaluated. FIG. 57 shows a protocol of the WT1-CTL induction test. FIG. 58 shows a method for preparing IL-4-DCs and HPL-IFN-DCs used in the WT1-CTL induction test. The IL-4-DCs collected on Day 7 were treated with the WT1-235 killer peptide at 100 μg/ml and 4° C. for 30 minutes and then subjected to the test (post-pulsed with WT1 peptides). Concerning HPL-IFN-DCs, the WT1-235 killer peptides were added to the maturation cocktails on Day 4, and the HPL-IFN-DCs collected on Day 5 were subjected to the test (pre-pulsed with WT1 peptides). FIG. 59 shows the results of evaluation of the proportion of WT1-CTL induction determined by the WT1-tetramer analysis. In comparison with existing IL-4-DCs, HPL-IFN-DC exhibited a higher WT1-CTL induction ability.

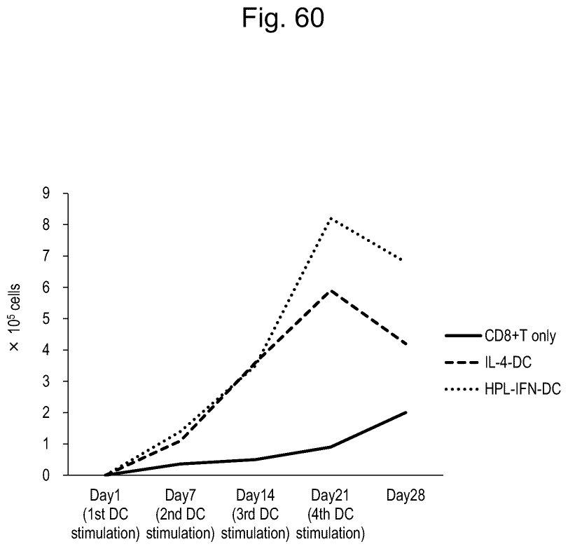

FIG. 60 shows the total number of WT1-CTLs induced by IL-4-DCs (post-pulsed with WT1 peptides) or HPL-IFN-DCs (pre-pulsed with WT1 peptides). The CD8⁺ T cells were stimulated 3 times using DCs (up to Day 21), and an increase in WT1-CTL was then observed. In comparison with IL-4-DCs, the level of induction was found to be higher in HPL-IFN-DCs. CD8⁺ T only was used as a negative control, which was not stimulated with DCs.

### INDUSTRIAL APPLICABILITY

The dendritic cells (DCs) prepared by the method of the present invention can be used for a method of dendritic cell therapy.

All publications, patents, and patent applications cited herein are incorporated herein by reference in their entirety.

1. A method for preparing cytotoxic dendritic cells from monocytes comprising performing non-adhesion culture of monocytes separated from the peripheral blood with the use of a serum-free medium containing a human platelet lysate (HPL), GM-CSF, and PEGylated interferon α and performing further non-adhesion culture with the addition of prostaglandin E2 and OK432.

2. The method for preparing dendritic cells from monocytes according to claim 1 comprising performing non-adhesion culture with the use of a serum-free medium containing a human platelet lysate (HPL), GM-CSF, and PEGylated interferon α for 2 to 5 days and performing further culture with the addition of prostaglandin E2 and OK432 for 1 to 2 days.

3. The method for preparing dendritic cells from monocytes according to claim 1 comprising culturing monocytes with the use of a serum-free medium containing a 1 to 10 (v/v) % human platelet lysate (HPL), 100 U/ml to 10,000 U/ml GM-CSF, 500 ng/ml to 5 μg/ml PEGylated interferon α, 5 ng/ml to 50 ng/ml prostaglandin E2, and 5 μg/ml to 50 μg/ml OK432.

4. The method for preparing dendritic cells from monocytes according to claim 1, wherein the serum-free medium is DCO-K.

5. The method for preparing dendritic cells from monocytes according to claim 1, wherein the viability of the dendritic cells is 90% or higher and the yield, which is the proportion of the number of the dendritic cells relative to the number of monocytes during culture, is 15% or higher.

6. The method for preparing dendritic cells from monocytes according to claim 1, wherein the dendritic cells are positive for CD14, CD16, CD56, CD83, CD86, CCR7 (CD197), HLA-ABC, and HLA-DR.

7. Dendritic cells obtained by the method for preparing dendritic cells from monocytes according claim 1.

8. A pharmaceutical composition comprising the dendritic cells according to claim 7.

9. The pharmaceutical composition according to claim 8, which has antitumor immune activity and can be used for cancer treatment.

10. A method for separating monocytes comprising culturing peripheral blood mononuclear cells in an adhesion culture vessel with the use of a serum-free medium containing a human platelet lysate (HPL) for 15 minutes to 3 hours, removing non-adherent cells, and collecting adherent cells.

11. The method for separating monocytes according to claim 10, which involves the use of a serum-free medium containing a 1 to 10 (v/v) % human platelet lysate (HPL).

12. The method for separating monocytes according to claim 10, wherein the serum-free medium is DCO-K.

13. The method according to claim 1 comprising adding a cancer-specific antigen to prepare dendritic cells having cancer-antigen-specific cytotoxic activity on dendritic cells.

14. Dendritic cells obtained by the method according to claim 13 having cancer-antigen-specific cytotoxic activity on dendritic cells.

15. A pharmaceutical composition containing the dendritic cells according to claim 14, which has antitumor immune activity and can be used for cancer treatment.
